(19) United States
(12) Patent Application Publication
(10) Pub. No.: US 2022/0062431 A1
(43) Pub. Date: Mar. 3, 2022

(54) COMPOUNDS COMPRISING STAPLED OR STITCHED PEPTIDES FOR IMPROVED DRUG DELIVERY

(71) Applicant: Sutura Therapeutics Ltd, Grace Bay, Providenciales (GB)

(72) Inventors: Keith Foster, Frimley, Surrey (GB); Adam James Reginald Gadd, Reading (GB)

(73) Assignee: Sutura Therapeutics Ltd, Grace Bay, Providenciales (GB)

(21) Appl. No.: 16/626,476

(22) PCT Filed: Jun. 28, 2018
(86) PCT No.: PCT/GB2018/051818

§ 371 Date: Dec. 24, 2019

(30) Foreign Application Priority Data
Jun. 28, 2017 (GB) 1710322.7

(51) Int. Cl. A61K 47/64; A61K 31/7105; A61K 47/54

(52) U.S. Cl. CPC A61K 47/64; A61K 47/54; A61K 47/545; A61K 31/7105

(57) ABSTRACT

The invention relates to improvements in drug delivery and to the use of Cell Penetrating Agents (CPA's) or Cell Penetrating Peptides (CPP's) which have been stabilized by, for example: i) stapling two amino acids to form Stapled CPP's (StaP's) or ii) stitching three or more amino acids to form stitched CPP's (StiP's). More particularly there is provided a drug carrying cell penetrating molecule (DCCPM) comprising: a biologically active compound (BAC), and a cell penetrating agent (CPA), which BAC and CPA are linked directly or via a bi-functional linker (BFL). The CPA is a stabilized peptide (CPP) which has a conformation imposed upon it by stapling to form a stapled peptide (StaP) or stitching to form a stitched peptide (StiP). The StiP or StaP comprise a cross link or bridge between at least two amino acids of the peptide and the cross link or bridge provides a cyclisation between at least two amino acids which are not formed by an olefin metathesis. Cyclisation may be achieved by one or more of: condensation of an aldehyde or ketone with a hydrazine or protected hydrazine; a thiol-ene Michael addition; a di-sulfide formation; a Huisgen 1,3 di-polar cycloaddition; a reaction between an amine and carboxylic acid; a singlet or triplet based carbine reaction; or a Suzuki or Sonogashira coupling.

### RELATED APPLICATION

This application is a national stage filing under 35 U.S.C. 371 of International Patent Application Serial No. PCT/GB2018/051818, filed Jun. 28, 2018, the entire content of which is incorporated herein by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on May 29, 2020, is named H066470098US00-SUBSEQ-DQP and is 5,858 bytes in size.

The present invention relates to improvements in drug delivery.

More particularly it relates to the use of Cell Penetrating Agents (CPA's), and more particularly still to the use of Cell Penetrating Peptides (CPP's) which have been stabilized by, for example: i) stapling two amino acids to form Stapled CPP's (StaP's) or ii) stitching three or more amino acids to form stitched CPP's (StiP's).

It differs from applicant's earlier patent application PCT/GB2016/054028 in using different chemistries to an olefin metathesis to stabilize the peptide. These chemistries provide a cross-link or bridge between at least two amino acids of the peptide, and the cross-link or bridge provides a cyclisation between the at least two amino acids. In these alternative chemistries cyclisation is achieved by one or more of:
i. condensation of an aldehyde or ketone with a hydrazine or protected hydrazine;
ii. a thiol-ene Michael addition;
iii. a di-sulfide formation;
iv. a Huisgen 1,3 di-polar cycloaddition;
v. a reaction between an amine and carboxylic acid;
vi. a singlet or triplet based carbine reaction; or
vii. a Suzuki or Sonogashira coupling.

These StaP's or StiP's may be composed of single or multiple staples or stitches and may be contiguous or non-contiguous along a peptide sequence.

These stabilized CPP's are conjugated to a drug or Biologically Active Compound (BAC) directly or via a Bi-Functional Linker (BFL) so that the BAC can be carried though a cell membrane by the CPP. The resulting molecules are referred to as Drug Carrying Cell Penetrating Molecules (DCCPM's).

The preferred BAC's delivered in this manner are oligonucleotides (ON's), more preferably still, electrically low charge carrying oligonucleotides (charge −3 to +3 at pH 7.5) and most preferably, electrically neutral oligonucleotides (charge −1 to +1 at pH 7.5), such as, but not limited to, peptide nucleic acids (PNAs), phosphorodiamidate morpholino oligonucleotides (PMO's) or modified derivatives thereof.

Alternatively, stabilised CPP's when conjugated to an ON, either directly or via a BFL, can associate covalently or non-covalently with a BAC that is either a DNA, RNA or protein molecule so that the BAC can be carried though a cell membrane by the CPP conjugated ON. The resulting molecules are referred to as Drug Targeting Cell Penetrating Molecules (DTCPM's).

The preferred BFL may be PEGylated, comprising poly ethylene glycol (PEG) groups including modifications such as an amine group, or incorporate a spacer, such as 3-Ala. These modifications can improve solubilisation or provide appropriate spacing between functional moieties.

The invention also relates to a method of facilitating the uptake of a BAC into a cell, the use of a DCCPM in the treatment of a disease requiring alteration of an endogenous or exogenous gene, a method of improving the bioavailability of a drug or BAC, a method of introducing a drug or BAC to a site which is refractory to the drug or BAC in its native state, a method of treating a subject comprising administering the DCCPM's of the invention and to a pharmaceutical composition comprising the DCCPM and pharmaceutical salts thereof, and one or more pharmaceutically acceptable excipients.

The invention also relates to a method of facilitating the uptake of a BAC that is either a DNA, RNA or protein molecule into a cell, the use of a DTCPM in the treatment of a disease requiring alteration of an endogenous or exogenous gene, a method of improving the bioavailability of a drug or BAC, a method of introducing a drug or BAC to a site which is refractory to the drug or BAC in its native state, a method of treating a subject comprising administering the DTCPM's of the invention and to a pharmaceutical composition comprising the DTCPM and one or more pharmaceutically acceptable excipients.

Still further aspects will be apparent from the detailed description.

### BACKGROUND TO THE INVENTION

In the treatment of all diseases it is desirable to deliver a drug or BAC into the body, and more preferably into a cell, at a target site, in a manner that ensures a maximal effect with minimal toxicity. This can be challenging.

An example of drugs or BACs which are delivered in a targeted manner are oligonucleotides (ON's), which term includes ON analogues.

ON's can target essential DNA, RNA and protein sequences and can modulate gene expression in a number of ways that includes steric blocking to suppress (i) RNA splicing, (ii) protein translation or (iii) other nucleic acid:nucleic acid or nucleic acid:protein interactions.

Specifically, the hybridisation of ON's to specific RNA sequence motifs prevents correct assembly of the spliceosome, so that it is unable to recognise the target exon(s) in the pre-mRNA and hence excludes these exons in the mature gene transcript. Exclusion of an in-frame exon can lead to a truncated yet functional gene product; exclusion of an out of frame exon results in a frame-shift of the transcript, potentially leading to a premature stop codon and a reduction in the target gene expression level. Similarly, ON's can alter cellular protein content via modulating RNA splicing to exclude specific exons of a gene from the mature mRNA transcript. Together, this has led to translational programs for conditions such as Duchenne muscular dystrophy (exon exclusion) and spinal muscular atrophy (exon inclusion)^{1,2} and subsequent market approval.

Additionally, ON's can be designed to target 5′ translation initiation start sites of viral gene transcript(s) to prevent binding of the translational machinery. Using antisense oligonucleotides (AO) to suppress viral translation is a well-established technology³ and has progressed into clinical trials for viral haemorrhagic fevers such as Marburg and Ebola^{4,5}. Although, AO can be designed to target 5′ translations initiation starts sites of endogenously expressed genes.

Also, ON can be designed to target 3′ untranslated region of an endogenous transcript that alters the nuclear export, translation and stability of the transcript. Such targets include, and are not limited to, poly adenylation and/or cleavage sites of the transcript.

Also, ON's can be designed to form aptamers such that the secondary and tertiary structures can bind proteins or other cellular targets thus impacting on specific gene expression levels or other cellular processes (e.g. post-translational modifications).

An advantage of steric blocking based suppression over that of siRNA/RNAi based RNase H-induction of the RNA Induced Silencing Complex is the reduced likelihood of off target side effects.

Modifications of an ON to produce a negatively charged backbone improve stability⁶⁻⁹, but these backbone chemistries e.g. 2′O-Methyl Phosphothioate analogues, elicit membrane toxicity issues, cause thrombocytopaenia and injection site problems upon clinical translation¹⁰, such that efficacy is prevented by toxicity issues, even when administration protocols become increasingly intermittent¹¹.

Indeed WO2013/150338 and WO2014/053622 both disclose delivering negatively charged ON's of small size (typically smaller than 1.5 KDa) by complexing them with positively charged linear or stapled peptides of equal or greater than 15 amino acids and in the range of 15-27 amino acids.

JACS, Vol 136, 2014, G J Hilinski et al, describe stapled and stitched peptides that are able to penetrate cells. Reference is made to the possibility that these peptides could be used to deliver an oligonucleotide, presumably in the same manner as disclosed in the international applications disclosed above, i.e. by complexation. There is nothing to suggest creating new entities of much larger size (greater than 1.5 KDa, through 2.5 KDa, 5 KDa, 7.5 KDa, 10 KDa, 12.5 KDa or more) by covalently linking a BAC with a CPA, optionally via a BFL and indeed, the prior methodology requires the respective components to have opposite charges to facilitate complexing.

The use of electrically low charge carrying oligonucleotides (charge −3 to +3 at pH 7.5) and most preferably electrically neutral oligonucleotides (charge −1 to +1 at pH 7.5), such as, but not limited to, peptide nucleic acids (PNAs), phosphorodiamidate morpholino oligonucleotides (PMO's), (covalently) conjugated directly or indirectly, using a BFL, was not apparent and indeed, limiting the charge on the ON further allows the use of smaller peptides (less than 15 amino acids in length, through 14, 13, 12, 11, 10, 9, 8, 7, 6 to as few as 5 or 4) as carriers.

The use of uncharged ON backbones, such as phosphorodiamidate morpholino oligonucleotides (PMOs), represent attractive BAC's as they have an impeccable safety record in a preclinical and clinical setting^{2,4,12-14}.

However, their ability to penetrate cells and access their targets is compromised due to their uncharged nature¹⁵.

Overcoming the problem of facilitating the entry of ON into cells is therefore desirable.

Other examples of drugs or BACs which are delivered in a targeted manner are DNA molecules (including linear and circularised molecules), RNA molecules and peptides.

Such strategies, including protein augmentation and viral mediated gene augmentation strategies have been the cornerstone of many medicines developments, however targeting issues and efficiencies of cargo delivery can constitute considerable problems.

Again, overcoming the problem of facilitating the entry of such DNA, RNA and peptides based BACs into cells is also desirable, e.g. circularised viral genomes in the absence of being packaged into infectious virions.

Over the last 20 years much research has been dedicated to developing CPA's that facilitate delivery of drugs and BAC's to the biological site of action.

The approach has generally been to use charged peptides as non-covalent complexes to facilitate cell entry of a BAC. Conjugation has also been tried.

WO2014/064258 is an example of the existing conjugating art. A negatively charged ON is coupled to a targeting peptide via a linker. The targeting peptide is a receptor targeting moiety, and not a stapled or stitched peptide, and indeed considerable doubt exists as to whether DNA or RNA molecules can gain cell entry using a receptor targeting moiety as once a negatively charged ON is bound to such a moiety, non-covalent interactions alter its conformation¹⁶.

WO89/03849 discloses oligonucleotide-polyamide conjugates. There is no disclosure of the use of stitched or stapled peptides. The methodology described uses oligonucleotides as a scaffold for the chain extension of peptides and not as a conjugate for delivery of a BAC, such as an ON, per se.

WO2011/131693 describes nucleic acid constructs which contain a nucleic acid specific for a given target gene and a selective inhibitor of a neurotransmitter transporter. There is no disclosure of the use of stitched or stapled peptides as a delivery agent.

WO2017/011820 describes cross linking groups used to stabilize peptides.

A peptide capable of effecting peptide-mediated cell delivery may also be referred to as a Cell Delivery Peptide (CDP). Examples include: poly arginine, penetratin (based upon an antennapedia homeodomain), or PMO internalization peptides (PIPs).

However, since their first description¹⁷ and given that many CPPs contain multiple arginines, β-alanine and 6-aminohexanoic acid residues, (e.g. poly-Arg12, TAT, Penetratin, Pip6a) [database maintained at http://crdd.osdd.net/raghava/cppsite/]¹⁸, it is surprising that no CPP-delivered drug has progressed through all phases of clinical trials. In part, this may be because the common arginine-rich core, which makes most CPP's effective, also causes membrane deformities¹⁹ and in higher mammals this manifests as prohibitive toxic side effects, such as tubular degeneration of the kidney²⁰.

At a physiological pH, and based on pKa of amino acid R groups, a formal charge (FC) can be calculated based on the formula:

$$FC = V - N - \frac{B}{2}$$

Where, V=valence electrons of the neutral atom in isolation; N=the number of non-bonding valence electrons on the defined atom; B=the total number of electrons shared in bonds.

Indeed, based on this, the CPPs typically used to date harbour many positively charged residues. It has been shown that there is a correlation between this positive charge and membrane toxicity²¹. In PCT/GB2016/054028, the applicant provided evidence that a reduction in formal charge of an 8 amino acid peptide from +3, to +2, +1 and 0 had interesting properties such that reduced charge variants retained cell entry properties. The applicant now bring forward data that reduced charged variants of CPP that have been cyclised by a ring closing metathesis have improved toxicological profiles when compared to the original CPP with a formal charge of +3.

Therefore, CPPs with a lower amount of positively charged residues within the amino acid sequence, whilst retaining the ability to cross a biological membrane, will be more clinically relevant. The applicant overcame this problem, as disclosed in PCT/GB2016/054028, and the present application is an extension thereof, which utilizes additional chemistries providing alternative stabilized peptides.

Previously, the applicant has illustrated this by delivering an ON targeted to repair a gene producing dystrophin. Targeting specific genes with ON is of course in itself known, as illustrated by, for example, WO2009/054725 and WO2010/123369. These publications however use a negatively charged backbone and deliver the cargo directly or using complexation.

PCT/GB2016/054028 taught preparing stapled and stitched peptides, two linked amino acids (stapled) or three or more linked amino acids (stitched), by incorporating amino acids into the peptide that are modified to bear e.g. an olefin (alkene) group (which may be incorporated at defined relative positions during solid-phase peptide synthesis). For example, on-resin ring-closing metathesis is then used to close one (stapled [denoted as StaP herein]) or two or more (stitched [denoted as StiP herein]) all-hydrocarbon cross-links that induce the peptide to adopt a stabilised structure, typically, but not essentially an alpha helix. For StaP's, it is preferred to use either one or both enantiomers of the un-natural amino acids, termed the S5 (S-pentenylalanine) or R5 (R-pentenylalanine), or the S8 (S-octenylalanine) or R8 (R-octenylalanine), depending on the stereo-chemical configuration. For StiP's, a further un-natural olefin-bearing α, α-di-substituted amino acid (B5 or B8) is utilised. Cross linking strategies are however not restricted to ring-closing metathesis of un-natural olefin-bearing α, α-di-substituted amino acids. Other cross-linking chemistry's may be used to stabilize the peptide, such as ring-closing metathesis between O-allylserine analogues (S-OAS or R-OAS).

In addition, it is envisioned that ON's can be designed to hybridise to a single stranded or double stranded DNA or RNA molecules (or analogues thereof) such that the hybridised DNA or RNA molecule can be delivered to a target cell. These DNA and RNA may be linear, branched, circularised or adopt any stable conformation. These DNA and RNA may be synthetic, engineered or natural molecules, e.g. circular DNA molecules generated during viral genome replication that creates an episome.

Stabilising peptides can be performed by crosslinking amino acids incorporated into defined relative position in the peptide sequence during solid phase synthesis or by chemically modifying existing amino acids. Cross-linking two specific amino acids (stapled) or cross-linking 3 or more amino acids (stitching) can be used to stabilize peptides of varying lengths^{22,23}. The specifically incorporated amino acids usually contain orthogonal functional groups allowing for specific and efficient cross-linking reactions to occur on resin or in solution post cleavage of the peptide. An examples of a cross-linking reaction based on resin ring closing metathesis of olefin baring amino acids is disclosed in PCT/GB2016/054028.

The present invention introduces additional CPPs based on alternative stapling or stitching technologies which introduce a cross link or bridge which provides a cyclisation between at least two amino acids. These include, but are not limited to:
a) the incorporation or derivatization of amino acids with functional groups for Huisgen 1,3 dipolar cyclo-additions, typically azidolysine and propargyl functional groups, such as, α-propargylalanine. Typically these reactions can be cyclized with a copper or a ruthenium based catalyst to yield 5 membered heterocycles, such as 1,2,3 triazoles with either 1,4 or 1,5 substitution favoured respectively. In addition to azide and propargyl functional groups, other substrates such as electron deficient nitriles and or diazoalkanes could be used²⁴; and
b) the use of protogenic amino acids for lactam formation by, for example, cyclizing lysine with a glutamic or aspartic acid residue within the peptide taking advantage of careful selection of protection groups during solid phase synthesis²⁵.

For persons skilled in the art, stapled CPPs can be developed based upon other cyclisation technologies. Combinations of stapled cyclisation based on a single methodology or several different crosslinking cyclisation methodologies could be utilized to form CPPs with either a single or multiple staples, either non-contiguously or contiguously distributed along a peptide. Further, persons skilled in the art can form StiP's that are similarly composed of one or more cyclisation technologies, including and not restricted to modular peptide components with differing orthogonal cyclisation chemistries.

The cellular entry dynamics of existing linear CPPs and the StiP's and StaP's differ. Traditional CPPs enter cells via energy-independent direct plasma membrane translocation or via energy-dependent, clathrin and caveolin-mediated endocytosis; whereas the StiP's and StaP's utilised in the invention enter via an energy dependent, but clathrin and caveolin independent mechanism^{22,26}. Given that StiP's and StaP's uptake is abrogated with reduced cellular decoration of heparin sulphate²² a macropinocytotic entry mechanism is inferred²⁷, suggesting this altered entry mechanism enables enhanced cellular uptake and bio-distribution compared to the state of the art.

Relative to their unmodified peptide precursors, StaP's and StiP's generally exhibit robust cellular uptake, significant resistance to proteolytic degradation, and in vivo stability that can support a half-life of more than 12 hours in non-human primates²⁸. It is likely that this increase in drug-likeness stems from the stabilised structure and the burial of the backbone amide bonds in the core of e.g. the α-helix. This structural rigidity also decreases the likelihood that StiP's and StaP's will be immunogenic, as the design of major histocompatibility complexes is such that peptides must adopt an extended conformation to be presented.

The importance of the stabilised CPP having a conformation imposed upon it should not be overlooked as the conformation imposed is responsible, in part, for the increased propensity for cellular uptake. The potential reduced or lack of membrane toxicity and immunogenicity enhances the clinical translatability of these compounds when conjugated to drugs and BAC's such as ON's.

The BAC and CPP can be covalently conjugated directly, or covalently conjugated via a BFL. Many functional groups may be used for conjugation reactions.

ONs can be used to induce a steric block to any gene in humans, animals and lower order organisms and thus can be applied to natural disease (including genetic and age-related diseases) or acquired diseases in humans and animals. Further, ONs can also be used to hybridise to a DNA or RNA molecule (or analogue), such as an episome, to facilitate its delivery, particular when conjugated to a StaP or StiP CPP.

As an example, viral haemorrhagic fevers (VHFs) are animal-borne illnesses in which a prolonged inflammatory cytokine response leads to the gradual destruction of veins and arteries. Causes of VHF include Ebola and Marburg viruses and several Arena viruses; these diseases are presently considered untreatable. Viral haemorrhagic fevers are characterized by high fever and bleeding disorders, and can cause death by shock and organ failure. AOs can be designed to target 5′ translation initiation start sites of viral gene transcript(s) to prevent binding of the translational machinery. Using AO to suppress viral translation is a well-established technology³ and has progressed into clinical trials for viral haemorrhagic fevers such as Marburg and Ebola^{4,5}. One PMO, AVI-7537 was evaluated for human use in the West African Ebola outbreak in 2014-15.

The use AOs have been adopted for other RNA steric block strategies. AOs can alter RNA splicing to either exclude an exon from the final processed mRNA, with Duchenne muscular dystrophy being the lead indication², or include an exon from the final processed mRNA, with Spinal Muscular Atrophy being the lead indication¹. Whilst both AO led to successful FDA approvals in 2016, systemic delivery of the respective AO's remains a considerable hurdle.

Some tissues are particularly refractory to naked PMO transfection, e.g. heart, which may reflect differential vesicle-mediated PMO uptake mechanisms²⁶. In fact, direct intra-cardiac injection of naked PMO does not even lead to efficient transfection²⁹, and refractory tissues tend to require repeat administration or high dose strategies³⁰⁻³². However, whilst CPP conjugation improves PMO bio-distribution and serum stability³³⁻³⁵, the toxicity associated with these linear, arginine rich peptides is still a major roadblock for pipeline development²⁰.

WO2016/187425 discloses an AO conjugated to a peptide that has been solely subject to cysteine arylation to form a bridge, no other technologies are disclosed. The cysteine arylation bridging technology is not a cyclisation technology, as it introduces rigid aromatic rings as the bridging moieties, thus it is not expected to have a stabilised conformation imposed upon it. Aside, the structure would not be stable following systemic administration due to the reduction of the thiol bonds which would release the bridge. In addition WO2016/187425 discloses arginine rich peptides only, therefore considerable membrane toxicity concerns remain for this technology.

Nitrogen arylation has been developed as a cyclisation technology³⁶. Although it is possible to conjugate such peptides to a BAC, these peptides would not be expected to form stabilized structure and importantly do no enter cells better than traditional linear peptides³⁶.

For effective clinical translation of steric blocking AOs, CPPs need to effectively deliver the BAC to either the cytoplasm or nucleoplasm whilst limiting any toxicity associated with cell entry.

Thus, providing DCCPM's or DTCPM's which are able to deliver a drug or BAC more efficiently or to a target site, or with lower toxicity and immunogenicity would be highly desirable.

### BRIEF SUMMARY OF THE DISCLOSURE

In accordance with a first aspect of the present invention there is provided a drug carrying cell penetrating molecule (DCCPM) comprising:
i. a biologically active compound (BAC), and
ii. a cell penetrating agent (CPA), which BAC and CPA are linked directly or via a bi-functional linker (BFL),
the CPA is a stabilized peptide (CPP) which has a conformation imposed upon it by stapling to form a stapled peptide (StaP) or stitching to form a stitched peptide (StiP), the StiP or StaP comprises a cross link or bridge between at least two amino acids of the peptide and the cross link or bridge provides a cyclisation between at least two amino acids which are not formed by an olefin metathesis.

By cyclisation is meant that a staple or stitch is formed directly between conformationally adjacent amino acids, as opposed to by the introduction of a separate “bridging molecule”, such as, for example, an aryl group, such as an aromatic ring or a perfluroaryl group. This direct cyclisation may be achieved by one or more of:
i. condensation of an aldehyde or ketone with a hydrazine or protected hydrazine;
ii. a thiol-ene Michael addition;
iii. a di-sulfide formation;
iv. a Huisgen 1,3 di-polar cycloaddition;
v. a reaction between an amine and carboxylic acid;
vi. a singlet or triplet based carbine reaction; or
vii. a Suzuki or Sonogashira coupling.

Particularly preferred cyclisations are obtained from chemistries iv) and v):

Using iv) a 5 membered heterocycle is formed between an azide and electron deficient nitrile containing amino acid or a propygyl containing amino acid.

Using v) a lactam is formed between a free amine containing amino acid and a carboxylic acid containing amino acid

A StaP may be formed by, for example, stapling two conformationally adjacent amino acids together, and a StiP may be formed by, for example, stitching at least three conformationally adjacent amino acids.

The stapling or stitching results in the formation of a cross link or bridge providing a cyclisation between two conformationally adjacent amino acids of the peptide.

In PCT/GB2016/054028 the cross link or bridge comprises two components, a hydrocarbon bridge and a terminal methyl group. The hydrocarbon bridge may be composed of a double hydrocarbon bond or a single hydrocarbon bond.

The present invention discloses a cross link or bridge comprised of two orthogonal functional groups able to from covalent bond or bonds between the two functional groups as illustrated in FIG. 1 (stapled peptides) and FIG. 2 (stitched peptides).

The CPP preferably comprises at least two of the following: un-natural amino acids, proteogenic amino acids or modified proteogenic amino acids bearing functional groups as illustrated in Table 1 below.

TABLE 1

| Unnatural or Modified Amino Acids | |
|---|---|
| 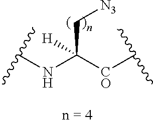 | 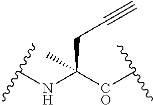 [SMILES: *N[C@@](C)(CC#C)C(*)=O] |
| 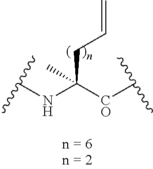 | 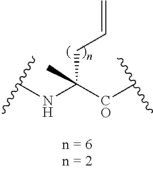 |
| 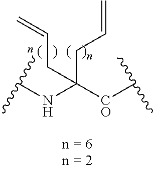 | 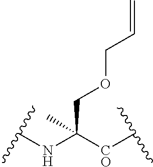 [SMILES: *N[C@@](C)(COCC=C)C(*)=O] |
| 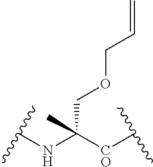 [SMILES: *N[C@](C)(COCC=C)C(*)=O] | |
| Proteogent Amino Acids | |
| 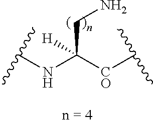 | 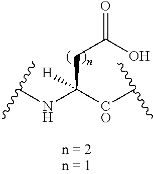 |

The preferred stapled or stitched CPPs incorporate one or more of the functional groups defined in Table 1, with an incorporated amino acid structure as shown in FIG. 3. In FIGS. 1, 2 and 3, X or X₁ illustrate a functional group, such as those in Table 1. R can be hydrogen or a moiety such as methyl or isobutyl designed to sterically constrain the geometry of groups from the α-carbon, or R can=X or X₁ as defined in Table 1.

In PCT/GB2016/054028 the CPP preferably comprises at least two un-natural amino acids bearing all-hydrocarbon tethers (e.g. α-methyl, α-pentenyl glycine) and the preferred stapled or stitched CPPs incorporate one or more of: a (S)-pentenylalanine (S5) or its enantiomer (R5), a S-octenylalanine (S8) or its enantiomer (R8) or combinations thereof (e.g. R-octenylalanine/S-pentenylalanine (R8/S5) or S-octenylalanine/R-pentenylalanine (S8/R5).

Alternative CPPs and their method of manufacture are disclosed in Chu et al, 2014 and associated supplementary information, and are incorporated by reference²².

The exemplified stabilized peptide comprises two or more orthogonal functional groups highlighted in Table 2 below that are covalently bound by the corresponding chemistry.

TABLE 2

| En- | | Reacting Functional | Resulting Functional |
|---|---|---|---|
| try | Functional group (X) | Group (X¹) | Group |
| 1 | 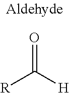 | 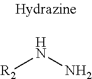 | 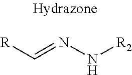 |
| 2 | 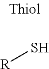 | 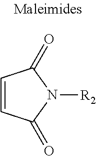 | 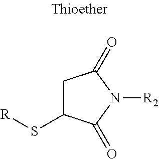 |
| 3 | 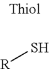 | 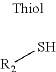 | 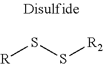 |
| 4 | 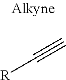 | 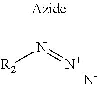 | 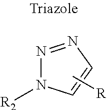 |
| 5 | 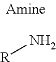 | 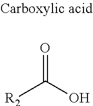 | 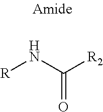 |
| 6 | 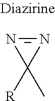 | Any | 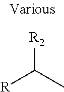 |
| 7 | 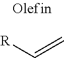 | 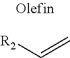 | 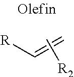 |
| | | | 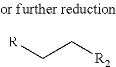 |
| 8 | Aryl or Olefin | Halide | R-R₂ |
| | R-M | R₂-X | |

The stabilized conformation typically comprises at least one alpha helix, extended 3₁₀-helix or poly (Pro) II helix. It may however, in the alternative, comprise at least one turn (for example, but not limited to, α, β, γ, δ or π), several turns to form a beta sheet or a hairpin, or a combination of one or more of: an alpha helix, extended 3₁₀-helix or poly (Pro) II helix, turn, beta sheet or hairpin.

The formal charge of a CPP is calculated at physiological pH (about 7.5) and is based on the pKa of amino acid R groups. These values (pKₓ) are represented in Table 3 below.

TABLE 3

| | 3- | 1- | | | | Residue | | | | |
|---|---|---|---|---|---|---|---|---|---|---|
| | Letter | Letter | Molecular | Molecular | Residue | Weight | | | | |
| Name | Symbol | Symbol | Weight | Formula | Formula | (—H₂O) | pK [symbol] | pK [symbol] | pK [symbol] | pl [symbol] |
| Alanine | Ala | A | 89.10 | C₃H₇NO₂ | C₃H₅NO | 71.08 | 2.34 | 9.69 | — | 6.00 |
| Arginine | Arg | R | 174.20 | C₆H₁₄N₄O₂ | C₆H₁₂N₄O | 156.19 | 2.17 | 9.04 | 12.48 | 10.76 |
| Asparagine | Asn | N | 132.12 | C₄H₈N₂O₃ | C₄H₆N₂O₂ | 114.11 | 2.02 | 8.80 | — | 5.41 |
| Aspartic acid | Asp | D | 133.11 | C₄H₇NO₄ | C₄H₅NO₃ | 115.09 | 1.68 | 9.60 | 3.65 | 2.77 |
| Cysteine | Cys | C | 121.16 | C₃H₇NO₂S | C₃H₅NOS | 103.15 | 1.96 | 10.28 | 8.18 | 5.07 |
| Glutamic acid | Glu | E | 147.13 | C₅H₉NO₄ | C₅H₇NO₃ | 129.12 | 2.19 | 9.67 | 4.25 | 3.22 |
| Glutamine | Gln | Q | 146.15 | C₅H₁₀N₂O₃ | C₅H₈N₂O₂ | 128.13 | 2.17 | 9.13 | — | 5.65 |
| Glycine | Gly | G | 75.07 | C₂H₅NO₂ | C₂H₃NO | 57.05 | 2.34 | 9.60 | — | 5.97 |
| Histidine | His | H | 155.16 | C₆H₉N₃O₂ | C₆H₇N₃O | 137.14 | 1.82 | 9.17 | 6.00 | 7.59 |
| Hydroxyproline | Hyp | O | 131.13 | C₅H₉NO₃ | C₅H₇NO₂ | 113.11 | 1.82 | 9.65 | — | — |
| Isoleucine | Ile | I | 131.18 | C₆H₁₃NO₂ | C₆H₁₁NO | 113.16 | 2.36 | 9.60 | — | 6.02 |
| Leucine | Leu | L | 131.18 | C₆H₁₃NO₂ | C₆H₁₁NO | 113.16 | 2.36 | 9.60 | — | 5.98 |
| Lysine | Lys | K | 146.19 | C₆H₁₄N₂O₂ | C₆H₁₂N₂O | 128.18 | 2.18 | 6.95 | 10.53 | 9.74 |
| Methionine | Met | M | 149.21 | C₅H₁₁NO₂S | C₅H₉NOS | 131.20 | 2.28 | 9.21 | — | 5.74 |
| Phenylalanine | Phe | F | 165.19 | C₉H₁₁NO₂ | C₉H₉NO | 147.16 | 1.83 | 9.13 | — | 5.48 |
| Proline | Pro | P | 115.13 | C₅H₉NO₂ | C₅H₇NO | 97.12 | 1.99 | 10.60 | — | 6.30 |
| Pyroglutamatic | Glp | U | 139.11 | C₅H₂NO₃ | C₅H₅NO₂ | 121.09 | — | — | — | 5.68 |
| Serine | Ser | S | 105.09 | C₃H₇NO₃ | C₃H₅NO₂ | 87.08 | 2.21 | 9.15 | — | 5.68 |
| Threonine | Thr | T | 119.12 | C₄H₉NO₃ | C₄H₇NO₂ | 101.11 | 2.09 | 9.10 | — | 5.60 |
| Tryptophan | Trp | W | 204.23 | C₁₁H₁₂N₂O₂ | C₁₁H₁₀N₂O | 186.22 | 2.83 | 9.39 | — | 5.89 |
| Tyrosine | Tyr | Y | 181.19 | C₉H₁₁NO₃ | C₉H₉NO₂ | 163.18 | 2.20 | 9.11 | 10.07 | 5.66 |
| Valine | Val | V | 117.15 | C₅H₁₁NO₂ | C₅H₉NO | 99.13 | 2.32 | 9.62 | — | 5.96 |

¹pK [symbol] is the negative of the logarithm of the dissociation constant for the —COOH group
²pK [symbol] is the negative of the logarithm of the dissociation constant for the — [symbol] H₃⁺ group
³pK [symbol] is the negative of the logarithm of the disassociation constant for any other group in the molecule
⁴pl is the pH at the [symbol] point
References D.R. Lids Handbook or Chemistry and Physics 72 Edition. CRC [symbol] Boca Raton, FL 1991. [symbol]
indicates data missing or illegible when filed

CPPs typically used to date harbour many positively charged residues. Reducing the amount of positively charged residues within the amino acid sequence, whilst retaining the ability to cross a biological membrane, will be more clinically relevant.

Accordingly, it is possible to reduce the charge on the peptide sequences.

The preferred BAC is an oligonucleotide (ON), more preferably still an anti-sense oligonucleotide (AO). Different anti-sense oligonucleotide chemistries are illustrated in Table 4 below, with the use of low charge or neutral charged chemistries, such as, phosphorodiamidate morpholino oligonucleotides (PMOs) being preferred.

TABLE 4

| | |
|---|---|
| 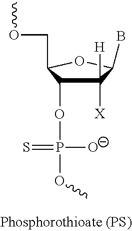 | 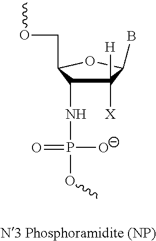 |
| 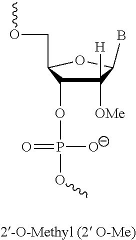 | 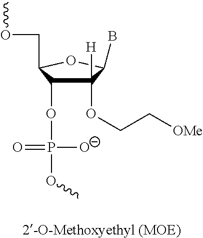 |
| 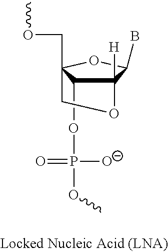 | 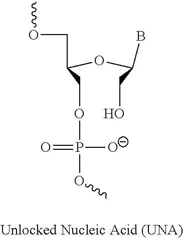 |
| 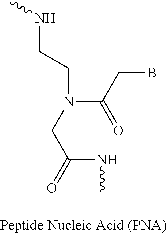 | 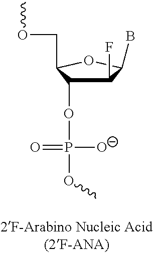 |
| 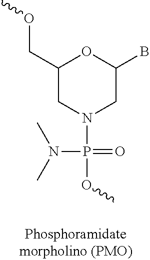 | 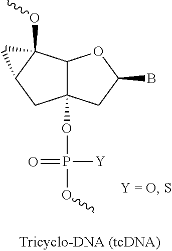 |

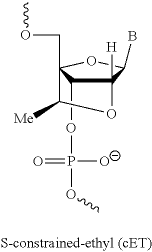

The BAC may target and alter the expression of an endogenous or exogenous gene. Endogenous gene targets include but are not limited to genes associated with neuromuscular disease, metabolic disease, cancer, age-related degenerative diseases, and exogenous gene targets include those of an acquired disease e.g. viral infections.

Whilst the BAC may be linked to the CPP directly (FIG. 4) the applicant has found the use of a BFL desirable. Exemplary, non-limiting BFL chemistries are illustrated in Table 5 below.

TABLE 5

| | | Linker | | |
|---|---|---|---|---|
| | | acronym | | |
| En- | | is | | |
| try | Linker (L) | present | Z | Y₃ |
| 1 | 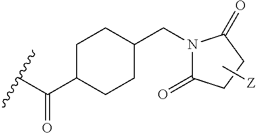 | SMCC | 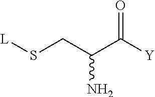 | 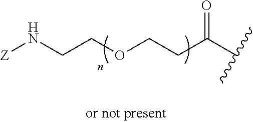 |
| 2 | 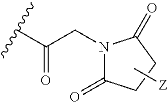 | AMAS | 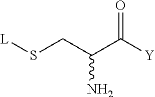 | 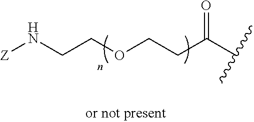 |
| 3 | 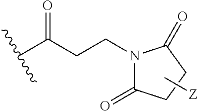 | BMPS | 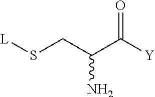 | 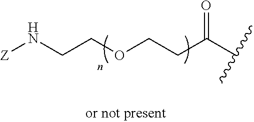 |
| 4 | 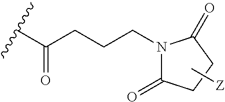 | GMPS | 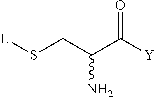 | 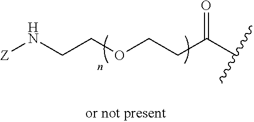 |
| 5 | 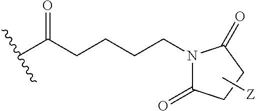 | DMVS | 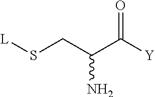 | 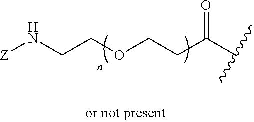 |
| 6 | 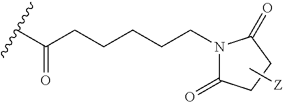 | EMCS | 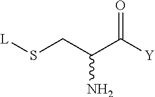 | 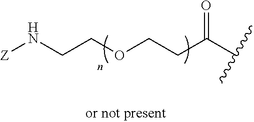 |
| 7 | 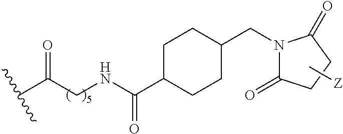 | LC- SMCC | 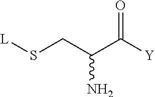 | 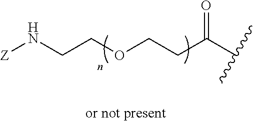 |
| 8 | 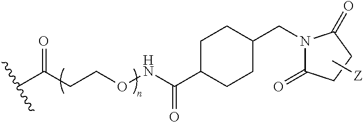 | SM (PEG)ₙ | 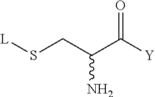 | 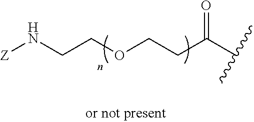 |
| 9 | 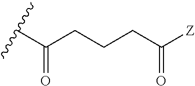 | DSG | Not present | 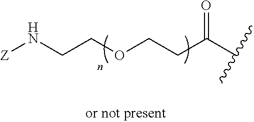 |
| 10 | 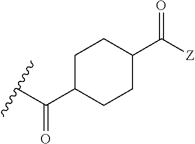 | DSCDS | Not present | 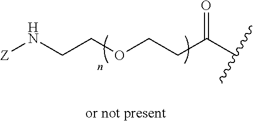 |
| 11 | 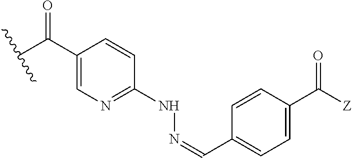 | HYNIC | Not present | 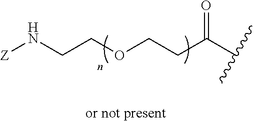 |

By way of a footnote to Table 5, the following should be noted:

FIGS. 5a and 5b and FIGS. 5c and 5d highlights general structure of a DCCPM where the following are preferred, but not limited to the following defined atoms or groups.

In a preferred embodiment illustrated in FIG. 5a, where Y1=Nitrogen, Y2=Hydrogen, Y3=spacer such as (PEG)n (n=5), but not limited to those identified in Table 5, Z=a sulfur containing moiety e.g. Cysteine and L=BFL such as SMCC (FIG. 5b).

Other embodiments may utilize variations over the structure shown in FIG. 5b. For example if another embodiment does not require a thiol for conjugation of the BFL to the CPA as illustrated in FIG. 5c, then Z=Y3, where Y3 is a spacer in Table 5. For a BFL that does not require a sulfur for conjugation of the BAC and CPA e.g. not limited to entries 9-11 in Table 5 Z=a covalent bond between L and Y3.

Other embodiments may not require the use of a spacer, a BFL and as such a thiol group for the formation of a DCCPM depicted in FIG. 4, then the following apply. The resin utilized in the solid phase synthesis of the peptide would be changed from rink amide or rink amide MBHA to 2-chlorotrityl resin to yield a peptide with a free carboxylic acid able to undergo coupling via standard peptide coupling conditions.

The orthogonal functional groups highlighted in Table 1 can also be used in a bio-conjugation reaction. These functional groups can be used to conjugate molecules to the DCCPM to provide desirable properties to the DCCPM. These will include, but are not limited to, an acetyl, a cholesterol, a fatty acid, a polyethylene glycol, a polysaccharide, an aminoglycan, a glycolipid, a phospholipid, a polyphenol, a nuclear localising signal, a nuclear export signal, an antibody, and a targeting molecule.

A preferred linker chemistry utilises an amine to sulfhydryl cross linker containing N-hydroxysuccinimide esters and malemide reactive groups separated by a cyclohexane spacer namely succinimidyl 4-(N-maleimidomethyl) cyclohexane-1-carboxylate (SMCC) to form a covalent bond between the BFL and the CPP and the BFL and BAC illustrated in FIG. 7a-d.

In a particularly preferred embodiment the linker may incorporate polyethylene glycol in single or multiple units (PEG)ₙ, where n=1 to 10 PEG molecules.

In the preferred embodiment of any of the above CPAs e.g. (FIG. 7a), may be covalently linked to a BFL, if required, preferentially incorporating (PEG)ₙ where n=1-10 linked to the N terminus of the peptide and terminated with a sulphur containing molecule e.g. cysteine (FIG. 7b). This allows further extension of the BFL with a bi-functional reactive molecule such as (SMCC) (FIG. 7c). This in turn is covalently linked to a functional group on the BAC, in the preferred embodiment a primary amine, thus generating a DCCPM (FIG. 7d).

Covalent linkage to the CPP may be via, for example, but not limited to, a β-ala or for someone skilled in the art any other suitable moiety which may include a branched or dendrimer like structure allowing for multiple BAC or CPPs to be conjugated.

In any particular embodiment the relative position of the cross-linking amino acids are referred to by their positioning within the sequence where the C terminus is the first amino acid referred to as positon 1 and subsequent amino acids are number in a N to C terminus fashion. A descriptor of the functionality is defined in Table 2. For example, a cross-link between a lysine reside (K) and glutamic acid reside (E) in the sequence RKF-[E-RLF-K] hitherto will be termed K1E5-8M (8M refers to an 8 mer amino acid), brackets in the sequence represents the cyclic portion of the peptide. Similarly for the sequence RKF—[S5-RLF-S5] cross linked between the S5 monomer by a ring closing metathesis will be referred to RCM1,5-8M.

In any particular embodiment the sequence of RKF-E-RLF-K will be a sequence of K1E5-8M not cyclized between the lysine and glutamic residue, hitherto referred to as K1E5-8M-NC.

In any particular embodiment where the relative spacing between the crosslinked amino acids is decreased, such as the sequence RKFR-[E-LF-K], will therefore be referred to as K1E4-8M; or relative spacing between the crosslinked amino acids is increased such RK-[E-FRLF-K] will be referred to as K1E6-8M.

Hereafter, where the CPP comprises the PEGylated sequence RKF-[E-RLF-K] and the BFL is a SMCC, the resultant compound is termed K1E5-CP8M (FIG. 7c).

According to a second aspect of the invention there is provided a method for facilitating the uptake of a biologically active compound (BAC) into a cell by the conjugation of the biologically active compound to a cell penetrating agent (CPA) which is a stabilized peptide that has a conformation imposed upon it by stapling to form a stapled peptide (StaP) or stitching to form a stitched peptide (StiP), the StiP or StaP comprises a cross link or bridge between at least two amino acids of the peptide and the cross link or bridge provides a cyclisation between the at least two amino acids which are not formed by an olefin metathesis, directly or via a bi-functional linker (BFL) to form a drug carrying cell penetrating molecule (DCCPM) and presenting said DCCPM to said cell in a suitable vehicle.

In another embodiment of any of the above CPAs e.g. (FIG. 8a), (PEG)ₙ is linked to the N terminus of the peptide and terminated with a hydrazynal nicotinic acid (HNA) (FIG. 8b). This allows conjugation of BFL with the appropriately modified BAC, in this instance the BAC is terminated with an aromatic aldehyde preferentially 4-formyl benzoic acid (FIG. 8c). This in turn is covalently linked to a functional group on the BAC to form a DCCPM (FIG. 8d).

Where the CPP comprises the sequence RKF-[E-RLF-K] and the BFL is a PEGylated hydrazynal nicotinic acid (HNA), the resultant compound is termed K1E5-HP8M (FIG. 8b).

Where the CPP comprises the sequence RKF-[E-RLF-K] and the BFL is SMCC with no PEGylation incorporated, the resultant compound is termed K1E5-C8M (FIG. 6d).

In any particular embodiment, a CPP derived from cyclizing lysine with a glutamic or aspartic acid residue can be directly conjugated to a BAC, such as an AO (FIG. 9).

In any particular embodiment, the method of forming a crosslink between two amino acid, modified amino acids or un-natural amino acids can be any and not limited to functional groups defined in Table 1.

In another preferred embodiment the orthogonal functional groups used for forming a cross-link are an incorporated azide residue and an alkyne residue.

Specifically in a cross link that used an azide and alkyne, the residues incorporated are (S)—N-Fmoc-2-(2′-propynyl)alanine baring an alkyne and (2S)—N-Fmoc-6-azido-hexanoic acid baring an azide.

In the event of incompatible synthesis or unfavorable cleavage condition of modified amino acids, some residues can be converted in situ, e.g. the treatment of lysine with N-diazo-1,1,1-trifluoromethanesulfonamide (TfN₃) to yield the azide functional group or the use of a di-azide molecule such as 1,3-benzenedicarbonyl di-azide or similar substituted di-azide to bridge between two propargyl containing amino acids.

In any particular embodiment the relative position of the cross linking amino acids are referred to by their positioning within the sequence and a descriptor of the functionality used and defined in table 1. Such as a cross-link between a (2S)—N-Fmoc-6-azido-hexanoic acid abbreviated to K(N₃) and (S)—N-Fmoc-2-(2′-propynyl)alanine abbreviated to B in the sequence RKF—[K(N₃)—RLF-B] will be referred to as B1K(N₃)5-8M (brackets in the sequence represents the cyclic portion of the peptide).

In any particular embodiment the sequence of RKF—K(N₃)—RLF-B will be a sequence of B1K(N₃)5-8M not cyclized between the azidolysine and propylgyl residue, will be hitherto referred to as B1K(N₃)5-8M-NC.

In any particular embodiment where the relative spacing between the amino acids is altered, this will be referred to as B1K(N₃)4-8M in case of a decrease (such as the sequence RKFR—[K(N₃)-LF-B]), or as B1K(N₃)6-8M in case of an increase (such as RK—[K(N₃)—FRLF-B]).

Hereafter, where the CPP comprises the sequence RKF—[K(N₃)—RLF-B] and the BFL is a PEGylated SMCC, the resultant compound is termed B1K(N₃)5-CP8M (FIG. 10c).

Where the CPP comprises the sequence RKF—[K(N₃)—RLF-B] and the BFL is a PEGylated hydrazynal nicotinic acid (HNA), the resultant compound is termed B1K(N₃)5-HP8M (FIG. 11b).

Where the CPP comprises the sequence RKF—[K(N₃)—RLF-B] and the BFL is SMCC with no PEGylation incorporated, the resultant compound is termed B1K(N₃)5-C8M.

As highlighted in FIG. 5d, a StaP or StiP conjugated ON (either with or without a BFL) can constitute a delivery vehicle for other BAC's. The ON could hybridise or associate with BAC's including DNA, RNA or protein molecules. The resulting molecules are referred to as Drug Targeting Cell Penetrating Molecules (DTCPM's). Of note, DTCPMs can hybridise to circular DNA or RNA molecules (episomes) or linear DNA or RNA molecules that are naturally generated during viral genome replication; in particular when viral packaging signals are not provided such that viral genomes are not packaged to form mature infectious virus particles.

According to a third aspect of the present invention there is provided a DCCPM or a DTCPM of the first aspect of the invention for use in the treatment of a disease requiring alteration of the expression of an endogenous or exogenous gene.

The DCCPM may be used in the treatment of, for example, a neuromuscular disease, metabolic disease, cancer, age-related degenerative disease or to treat an acquired infection.

The DTCPM may be used in the treatment of, for example, a neuromuscular disease, metabolic disease, cancer, age-related degenerative disease or to treat an acquired infection.

In one embodiment the DCCPM is used in the treatment of a muscular dystrophy e.g. Duchenne muscular dystrophy (DMD) although the skilled person will readily appreciate that the invention can be used to target a wide range of genes.

In the case of DMD the DCCPM may comprise an AON targeting exon 51 of the dystrophin gene.

In accordance with a fourth aspect of the present invention there is provided a method of improving the bioavailability of a drug or BAC, comprising linking the drug or BAC to a CPP which is a stabilized peptide which has a conformation imposed upon it by stapling to form a stapled peptide (StaP) or stitching to form a stitched peptide (StiP) and the StiP or StaP comprises a cross link or bridge between at least two amino acids of the peptide and the cross link or bridge provides a cyclisation between the at least two amino acids which are not formed by an olefin metathesis.

The shape imposed by the stabilization may generate a structure with a minimized free energy conformation.

In accordance with a fifth aspect of the present invention there is provided a method of introducing a drug or BAC to a site which is refractory to a drug or BAC in its native state, comprising linking the drug or BAC to a CPP which is a stabilized peptide which has a conformation imposed upon it by stapling to form a stapled peptide (StaP) or stitching to form a stitched peptide (StiP). The StiP or StaP comprises a cross link or bridge between at least two amino acids of the peptide and the cross link or bridge provides a cyclisation not formed by an olefin metathesis and administering it to a subject.

The DCCPMs of the invention can be used to administer the drug or BAC to a target tissue, such as, for example the heart, brain or muscle.

In accordance with a sixth aspect of the present invention there is provided a method of treating a subject to alter the expression of an endogenous or exogenous gene comprising administering a DCCPM or DTCPM of the invention to a subject.

In accordance with a seventh aspect of the present invention there is provided a composition comprising a DCCPM or DTCPM of the invention and one or more pharmaceutically acceptable excipients enabling the composition to be administered orally, parenterally, intravenously or topically.

### BRIEF DESCRIPTION OF THE DRAWINGS

Embodiments of the invention are further described hereinafter with reference to the accompanying drawings, in which:

FIGS. 1a-c illustrate examples of un-cyclised CPPs (left) and some of the resulting stabilized structures (right) that can be formed therefrom;

FIGS. 2a-d illustrate examples of stitched CPA's: FIG. 2a is formed by cyclizing two sets of orthogonal amino acid that are consecutively placed in the peptide sequence; FIG. 2b illustrates stitched CPA's formed by cyclizing two different sets of orthogonal amino acids not consecutively placed in the peptide sequence; FIG. 2c illustrates a stitched CPA where two sets of amino acids to be cross-linked originate from one amino acid; and FIG. 2d illustrates two alternative sets of amino acids that originate form one amino acid that can be cross linked to form a continuous stitched CPA;

FIG. 3 exemplifies the structure of an amino acid that can be incorporated into CPPs with varying functional groups as defined in Table 1. The functional groups presented by X and X₁ can be cross-linked to form CPAs and can also be used for bio-conjugation;

FIG. 4 illustrates the direct conjugation of a stabilized CPP to a BAC without the use of BFL;

FIG. 5 illustrates the connectivity of BFLs and overall structure of DCCPM and DTCPM.

FIG. 5a is a schematic diagram of a DCCPM illustrating the connectivity of BFL with non-limiting examples from Table 5 where the BAC is an antisense oligonucleotide (AO) or an oligonucleotide (ON);

FIGS. 5b-c are schematic diagrams of a DCCPM illustrating the connectivity of BFL with non-limiting examples from Table 5 where the BAC is an AO or an ON where the CPA has been stabilized by a either a 1,4 substituted or a 1,5 substituted 1,2,3 triazole;

FIG. 5d is a schematic diagram of a DTCPM illustrating the connectivity of BFL with non-limiting examples from Table 5 where oligonucleotide constitute part of the delivery molecule; a non-limiting list of BAC's delivered by this technology includes DNA, RNA or protein molecules (in effect the ON is part of the BFL and connects to the BAC);

FIG. 6 illustrates some preferred embodiments of DCCPMs incorporating different BFLs.

FIG. 6a is a general schematic of a DCCPM and illustrates connectivity of BFL defined but not limited to Table 5;

FIG. 6b is a schematic diagram illustrating a preferred embodiment of an DCCPM with an lactam stabilized CPP with an incorporated PEG₅ which is terminated with a thiol containing molecule (cysteine) and conjugated to the BAC using the heterobifunctional protein cross linker Succinimidyl-4-[N-maleimidomethyl] cyclohexane-1-carboxylate (SMCC);

FIG. 6c is a schematic diagram illustrating a preferred embodiment of a DCCPM with an lactam stabilized CPP with an incorporated PEG₅ which is terminated with a hydryzynal nicotinic acid (HNA) and subsequently conjugated to a BAC which has been modified with 4-formyl benzoic acid;

FIG. 6d is a schematic diagram illustrating a preferred embodiment of an DCCPM with an lactam stabilized CPP which is terminated with a thiol containing molecule (cysteine) and conjugated to the BAC using the heterobifunctional protein cross linker Succinimidyl-4-[N-maleimidomethyl] cyclohexane-1-carboxylate (SMCC);

FIG. 7 illustrates some preferred embodiments of DCCPMs incorporating CPPs stabilized by crosslinking lysine and glutamic residues forming a lactam;

FIG. 7a is a schematic of a CPP stabilized by cyclisation between two proteogenic amino acids to form a lactam;

FIG. 7b illustrates the incorporation of PEG₅ to the N terminus of the CPA and its termination with a thiol containing molecule (cysteine);

FIG. 7c illustrated the incorporation of the hetero bi-functional linker SMCC into the DCCPM;

FIG. 7d is a schematic diagram illustrating a preferred embodiment of a DCCPM with an K-E stabilized CPP with an incorporated PEG₅ which is terminated with a thiol containing molecule (cysteine) and subsequently conjugated to a BAC using the heterobifunctional protein cross linker Succinimidyl-4-[N-maleimidomethyl] cyclohexane-1-carboxylate (SMCC);

FIG. 8 illustrates some preferred embodiments of DCCPMs incorporating CPPs stabilized by crosslinking lysine and glutamic residues forming a lactam;

FIG. 8a is a schematic of a CPP stabilized by cyclisation between two proteogenic amino acids to form a lactam;

FIG. 8b is a schematic diagram illustrating a preferred embodiment of an DCCPM with an lactam stabilized CPP with an incorporated PEG₅ which is terminated with a hydrazynal nicotinic acid (HNA) and subsequently conjugated to a 4-formyl benzoic modified BAC;

FIG. 8c a schematic diagram of a 4-formyl benzoic modified BAC;

FIG. 8d is a schematic diagram illustrating a preferred embodiment of a DCCPM with an lactam stabilized CPP with an incorporated PEG₅ which is terminated with a hydryzynal nicotinic acid (HNA) and subsequently conjugated to a BAC which has been modified with 4-formyl benzoic acid;

FIG. 9 is a schematic diagram illustrating a preferred embodiment of a directly conjugated lactam containing CPP to a BAC;

FIG. 10 are schematic diagrams illustrating the conjugation of a 1,4 substituted triazole stabilised CPP to a BAC to form a preferred embodiment of a DCCPM;

FIG. 10a is a schematic diagram of a 1,4 substituted triazole stabilised CPP;

FIG. 10b is a schematic diagram of a 1,4 substituted triazole stabilised CPP with an incorporated PEG₅ and terminated with a thiol-containing molecule (cysteine);

FIG. 10c is a schematic diagram of a 1,4 substituted triazole stabilised CPP and illustrates the incorporation of the heterobifunctional protein cross linker Succinimidyl-4-[N-maleimidomethyl] cyclohexane-1-carboxylate (SMCC);

FIG. 10d is a schematic diagram illustrating a 1,4 substituted triazole stabilized CPP as part of a DCCPM. It should be noted that the 1,2,3 triazole could contain other isomers e.g. 1,5 substituted triazole;

FIG. 11a illustrates connectivity of 1,4 substituted triazole containing CPP;

FIG. 11b is a schematic diagram illustrating a preferred embodiment of a DCCPM with a triazole stabilized CPP with an incorporated PEG₅ which is terminated with a hydrazynal nicotinic acid (HNA) and subsequently conjugated to a 4-formyl benzoic modified BAC;

FIG. 11c a schematic diagram of a 4-formyl benzoic modified BAC;

FIG. 11d is a schematic diagram illustrating a preferred embodiment of a DCCPM with a 1,4 substituted triazole stabilized CPP with an incorporated PEG₅ which is terminated with a hydryzynal nicotinic acid (HNA) and subsequently conjugated to a BAC which has been modified with 4-formyl benzoic acid;

FIG. 12a is circular dichroism spectra of FITC-K1E4-CP8M, FITC-K1E5-CP8M and FITC-K1E6-CP8M blanked to D2O and collected at room temperature and in triplicate. FITC-K1E4-CP8M and FITC-K1E5-CP8M displays random coils and FITC-K1E6-CP8M exhibits a degree of α-helicity.

FIG. 12b is circular dichroism spectra of K1E4-CP8M, K1E5-CP8M and K1E6-CP8M blanked to D2O and collected at room temperature and in triplicate. K1E5 displays a random coil, where as K1E4 and K1E6 show extended Pi helices;

FIG. 13 is flow cytometry data for HeLa pLuc 705 cells incubated in the presence of FITC labeled CPAs;

FIG. 13a(i) is a comparison of FL1+ cells of HeLa pLuc 705 cells incubated with K1E4-P8M-FITC or K1E4-P8M-FITC-NC 4 hours n=4, error bars show standard deviation 2 way ANOVA was performed and showed no interaction affect. Data illustrates that there was no significant difference in the number of FL1 positive cells when K1E4-P8M-FITC was cyclized;

FIG. 13a(ii) is a comparison of FL1+ cells of HeLa pLuc 705 cells incubated with K1E5-P8M-FITC or K1E5-P8M-FITC-NC 4 hours n=4, error bars show standard deviation 2 way ANOVA was performed and showed no interaction affect. Data illustrates that there was no significant difference in the number of FL1 positive cells when K1E5-P8M-FITC was cyclized;

FIG. 13a(iii) is a comparison of FL1+ cells of HeLa pLuc 705 cells incubated with K1E6-P8M-FITC or K1E6-P8M-FITC-NC for 4 hours. n=4, error bars show standard deviation 2 way ANOVA was performed * represents p<0.05, ** represents p<0.001. Data illustrates that there is a significant increase in the number of FL1 positive cells at a given concentration when K1E6-P8M-FITC is cyclized;

FIG. 13b(i) is a comparison of MFI of HeLa pLuc 705 cells incubated with K1E4-P8M-FITC or K1E4-P8M-FITC-NC for 4 hours. n=4, error bars show standard deviation. 2 way ANOVA was performed and showed no interaction affect;

FIG. 13b(ii) is a comparison of MFI of HeLa pLuc 705 cells incubated with K1E5-P8M-FITC or K1E5-P8M-FITC-NC for 4 hours. n=4, error bars show standard deviation. 2 way ANOVA was performed and showed no interaction affect;

FIG. 13b(iii) is a comparison of MFI of HeLa pLuc 705 cells incubated with K1E6-P8M-FITC or K1E6-P8M-FITC-NC for 4 hours. n=4, error bars show standard deviation. 2 way ANOVA was performed * represents p<0.05;

FIG. 13c is a comparison % viable cells judged by gated cells compared to negative controls of HeLa pLuc 705 cells incubated with (i) K1E4-P8M-FITC versus K1E4-P8M-FITC-NC, (ii) K1E5-P8M-FITC versus K1E5-P8M-FITC-NC and (iii) K1E6-P8M-FITC versus K1E4-P8M-FITC-NC for 4 hours. n=4, error bars show standard deviation. Data shows that there is no significant difference in the % of cells recovered when normalized to untreated controls;

FIG. 14 is a Micrograph of PC3-705 cells treated with PMO conjugated to either K1E4, K1E5 or K1E6 CP8M. Cells were treated to for 4 hours without transfection reagents then images captured. Images were captured using either brightfield objective or using epifluorescence to show FITC labeled PMO. Images were subsequently merged to show cellular localization. There is significant intracellular PMO signal evident in PC3-705 cells treated with PMO conjugated to either K1E4, K1E5 or K1E6 CP8M only. Unconjugated PMO gives no intracellular signal;

FIGS. 15a-c are flow cytometry data for HeLa pLuc 705 cells incubated in the presence of FITC labeled CPAs;

FIG. 15a is a comparison of FL1+ cells of HeLa pLuc 705 cells incubated with RCM1,5-P8M-FITC-3+, RCM1,5-P8M-FITC-2+, RCM1,5-P8M-FITC-1+, RCM1,5-P8M-FITC-0+, for 4 hours. n=5, error bars show standard deviation. * represents p<0.05, ** represents p<0.001. *a represents an interaction between RCM1,5-P8M-FITC-1+ and all other groups, *b represents an interaction between RCM1,5-P8M-FITC-0+ and all other groups. Data shows that RCM1,5-P8M-FITC-1+ has a significant reduction in FL1+ cells compared to other treatments over a range of concentrations. RCM1,5-P8M-FITC-0+ shows in increase in the number of FL1+ cells over a range of concentrations compared to other treatments;

FIG. 15b is a comparison of MFI of HeLa pLuc 705 cells incubated with RCM1,5-P8M-FITC-3+, RCM1,5-P8M-FITC-2+, RCM1,5-P8M-FITC-1+, RCM1,5-P8M-FITC-0+ for 4 hours. n=5, error bars show standard deviation. * represents p<0.05, ** represents p<0.001. There is no significant difference in the MFI between RCM, 1,5-P8M-FITC-0+, 1+ and 2+ at a given concentration. However RCM1,5-P8M-FITC-3+ shows an increase in MFI at higher concentrations >1 μM;

FIG. 15c is a comparison of % viable cells judged by gated cells compared to negative controls of HeLa pLuc 705 cells incubated with RCM1,5-P8M-FITC-3+[RKF(S5RLFS5)], RCM1,5-P8M-FITC-2+[RIF(S5RLFS5)], RCM1,5-P8M-FITC-1+[IIF(S5RLFS5)], RCM1,5-P8M-FITC-0+[IIF(S5ILFS5)] for 4 hours. n=3, error bars show standard deviation * represents p<0.05. *a represents an difference between RCM1,5-P8M-FITC-3+ and RCM1,5-P8M-FITC-2+, *b represents an difference between RCM1,5-P8M-FITC-2+ and RCM1,5-P8M-FITC-0+ and *c represents an difference between RCM1,5-P8M-FITC-0+ and RCM1,5-P8M-FITC-3+. Data shows that at highest concentration 100 μM a significant loss in the number of viable cells recovered when comparing RCM1,5-CP8M-FITC-3+ to RCM1,5-CP8M-FITC-2+ and an increase in the number of cells when comparing RCM1,5-CP8M-FITC-2+ to RCM1,5-CP8M-FITC-0+. Similarly at 33 μM RCM1,5-CP8M-FITC-3+ compared to RCM1,5-CP8M-FITC-2+ and RCM1,5-CP8M-FITC-0+ showed a decrease in the % viable cells. At all other concentration no other significant affects were observed apart from at 0.05 μM. At 0.05 μM RCM1,5-CP8M-FITC-0+ showed an increase in viable cells over RCM1,5-CP8M-FITC-3+ and RCM1,5-CP8M-FITC-2;

FIG. 16 illustrates % cell viability of HeLa pLuc 705 cells when comparing treatment with RCM1,5-P8M-3+-FITC to K1E5-P8M-FITC for 4 hours. n=3-4, error bars show standard deviation * represents p<0.001. At all concentrations an increase in viability was observed when comparing K1E5-P8M-FITC to RCM1,5-P8M-FITC-3+.

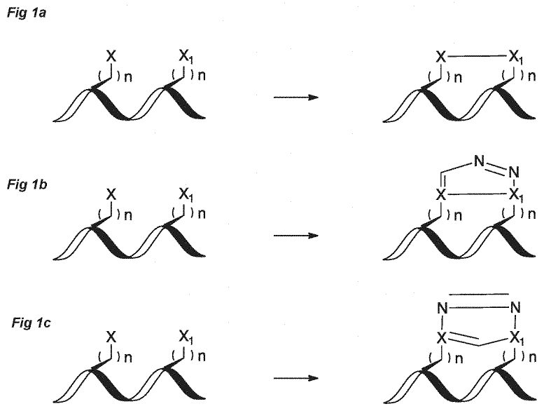

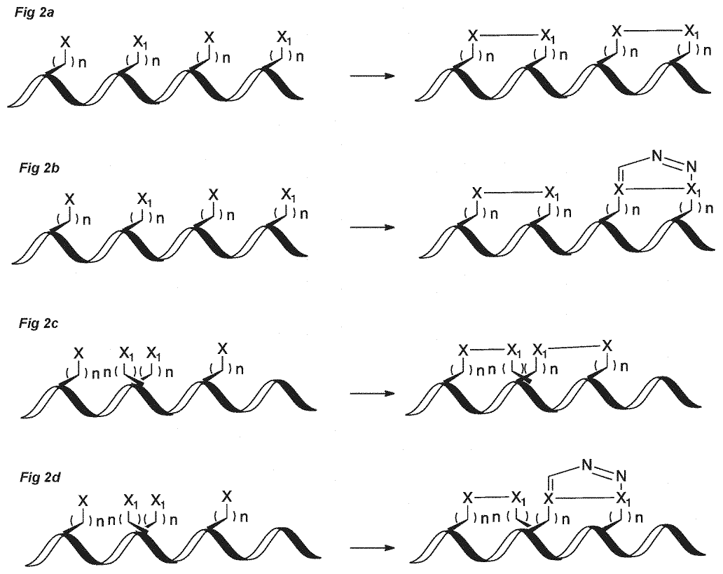

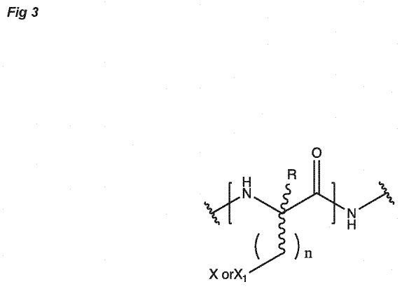

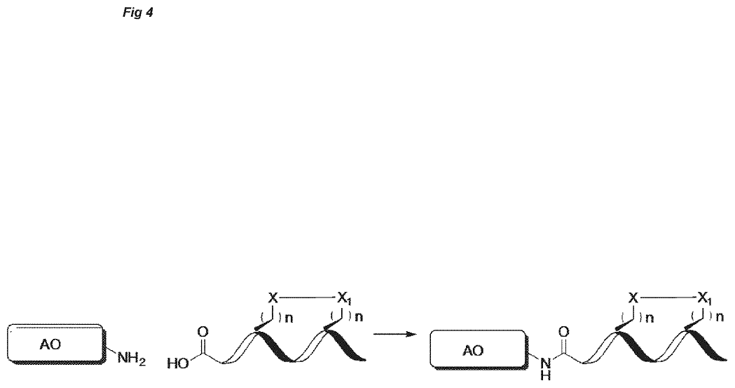

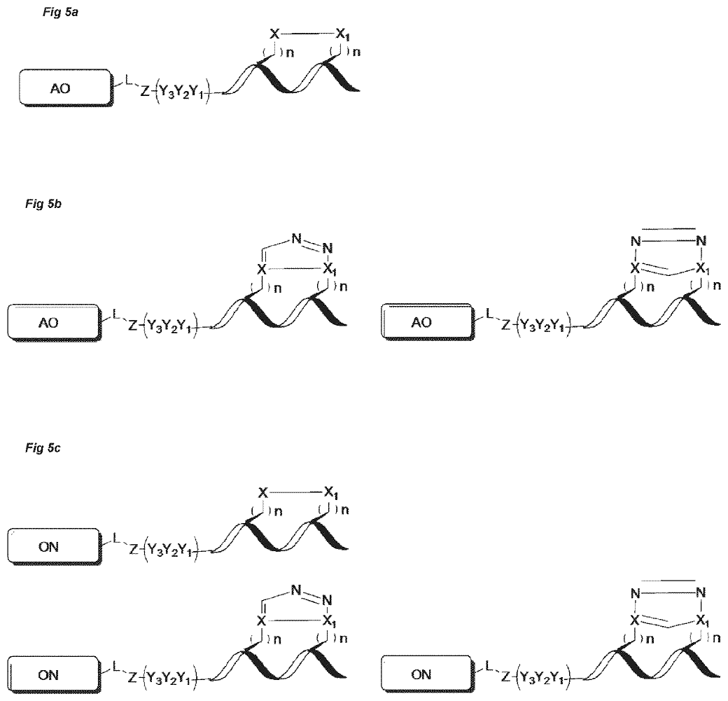

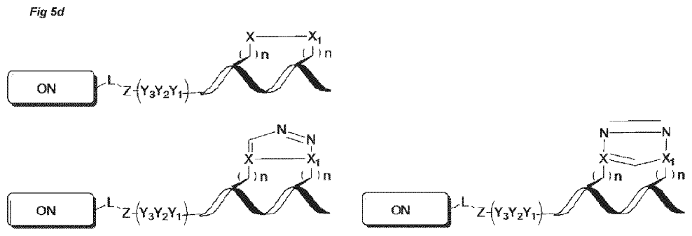

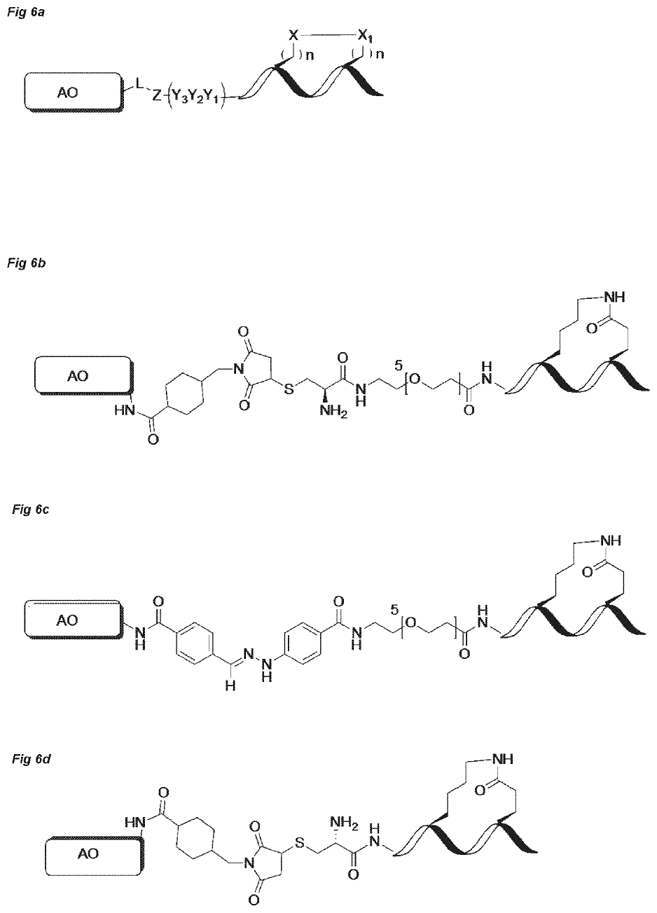

### DETAILED DESCRIPTION

The invention demonstrates that different stapling and stitching chemistries (to that disclosed in applicants earlier application PCT/GB2016/054028) can be used to produce drug carrying cell penetrating molecule (DCCPM) and that these stabilized peptides can confer different and potentially beneficial effects such as, for example, lower toxicity.

An exemplary drug carrying cell penetrating molecule (DCCPM) was produced with a FITC label in order to demonstrate cellular uptake.

The exemplary DCCPM comprises:
i) a biologically active compound (BAC);
ii) a cell penetrating agent (CPA) which is a stabilized peptide; and
iii) a bi-functional linker (BFL).

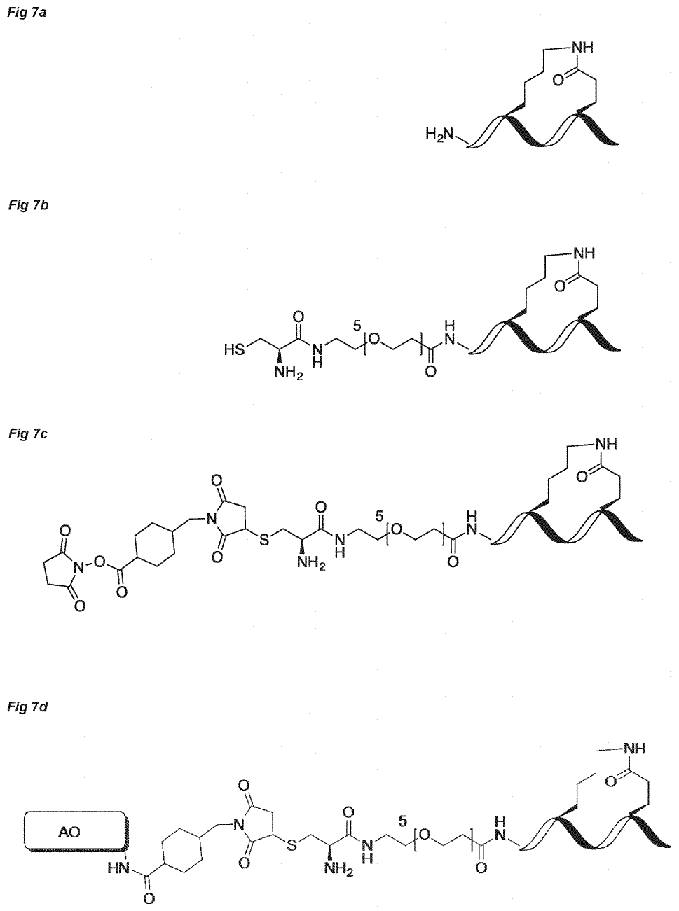

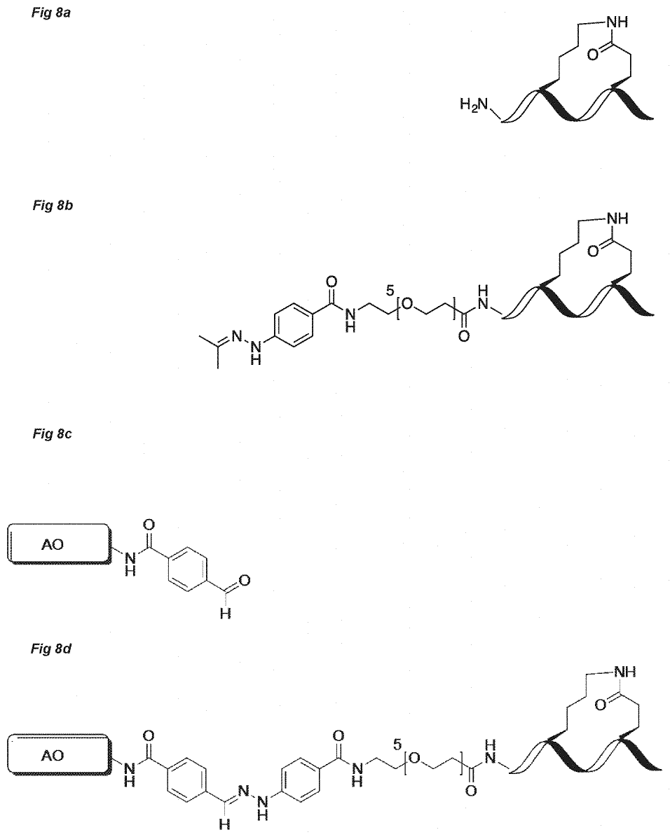

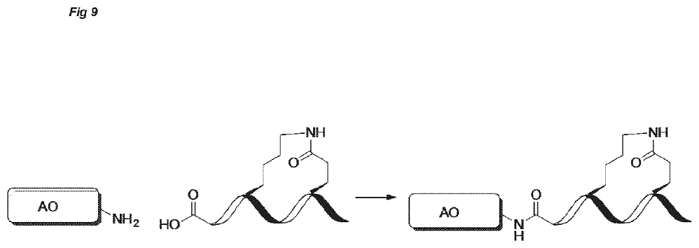

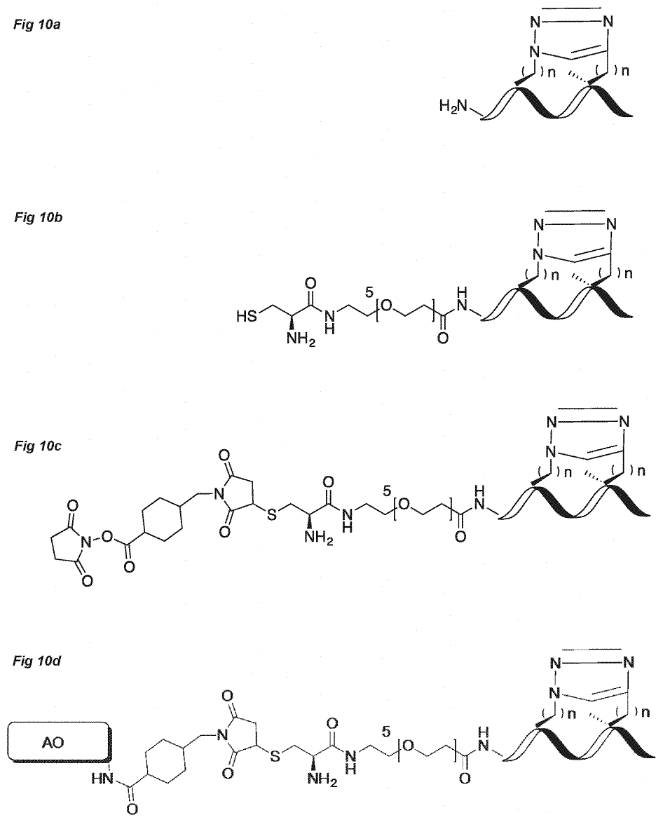

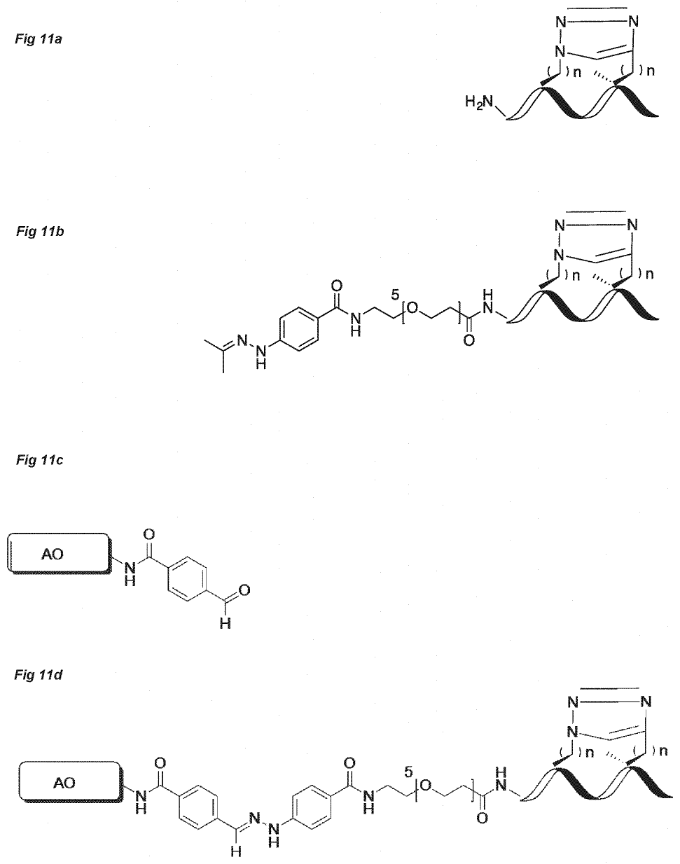

The three components forming the DCCPM are described in more detail below, and particularly favored embodiments are illustrated by way of reference to FIGS. 7a-d and FIGS. 8a-d.

### 1. The Biologically Active Compound

The biologically active compound is any compound that can exert a biological effect within a biological cell. Preferably, though not essentially, the BAC is one which will impact on the expression of one or more endogenous or exogenous genes. Examples include nucleic acids, DNAzymes, ribozymes, aptamers and pharmaceuticals. Preferred biologically active compounds for use in the present invention include electrically neutral oligonucleotides (charge −1 to +1 at physiological pH—about 7.5) such as peptide nucleic acids (PNAs) or PMOs or their modified derivatives that might impart a small electric charge (either positive or negative).

The biologically active compound may be used as a steric blocking compound to suppress or enhance: i) RNA splicing; ii) protein translation or iii) other nucleic acid:nucleic acid or nucleic acid:protein interactions, altering the gene expression of endogenous or exogenous (pathogen derived) genes.

The hybridisation of ON's to specific RNA sequence motifs prevents correct assembly of the spliceosome, so that it is unable to recognise the target exon(s) in the pre-mRNA and hence excludes these exon in the mature gene transcript. Exclusion of an in-frame exon can lead to a truncated yet functional gene product; exclusion of an out of frame exon results in a frame-shift of the transcript, potentially leading to a premature stop codon and a reduction in the target gene expression level.

Additionally, ON's can be designed to target 5′ translation initiation start sites of endogenous or viral gene transcript(s) to prevent binding of the translational machinery. Using AO to suppress viral translation is a well-established technology and has progressed into clinical trials for viral haemorrhagic fevers such as Marburg and Ebola.

Also, ON can be designed to target 3′ untranslated region of an endogenous transcript that alters the nuclear export, translation and stability of the transcript. Such targets include, but are not limited to polyadenylation and/or cleavage sites of the transcript.

Also, ON can be designed to form aptamers such that the secondary and tertiary structures can bind proteins or other cellular targets thus impacting on specific gene expression levels.

Non-limiting exemplary ON chemistries are illustrated in Table 4.

In the non-limiting example illustrated, the target is exon 51 of the dystrophin gene and comprises the sequence:

Sequence id 1:
5′ CUCCAACAUCAAGGAAGAUGGCAUUUCUAG 3′

### 2. The Cell Penetrating Agent (CPA) which is a Stabilized Peptide

The cell penetrating agents of the invention are stabilized peptides.

The peptides may be stabilized by cross linking of 2 amino acids, modified amino acids or un-natural amino acids, to form a stapled peptide (StaP) or crosslinking 3 or more residues to form a stitched peptide (StiP).

Crosslinking by stapling and stitching may confer a property, e.g. a solvated conformation such as, but not limited to, an alpha helix, extended 3₁₀-helix or poly (Pro) II helix, a turn (for example, but not limited to, α, β, γ, δ or π), several turns to form a beta sheet or a hairpin, or a combination of one or more of: an alpha helix, extended 3₁₀-helix or poly (Pro) II helix, turn, or beta sheet, an energetic conformational bias dependent on solvation environment e.g. interaction with plasma membrane, cellular penetrance, and biological activity.

Non-limiting examples of alternative chemistries to that described in PCT/GB2016/054028 for producing StaP and StiP are illustrated in Table 6 and include peptide sequences with nominal position for cross linking by amino acid, modified amino acids or unnatural amino acids illustrated by X and refereeing to functional groups defined but not limited to Table 1.

Stabilisation of peptides e.g. stitching or stapling, can be performed by a variety of means dependent on the functional group incorporated into the peptide. Non-limiting examples of functional groups are demonstrated in Table 2. Some reactions require catalyst or have preferential reagents for stabilization and are illustrated in Table 6 below.

TABLE 6

| Table 1 | | Catalyst or |
|---|---|---|
| Entry | Brief description | desirable reagent |
| 1 | Condensation of aldehyde or | Aniline as a catalyst |
| | ketone with hydrazine or | |
| | protected hydrazine | |
| 2 | Thiol-ene Michael addition | Reducing agent such a |
| | | immobilized TCEP or suitable |
| | | base or nucleophile. |
| 3 | Di-sulfide formation | Reducing agent such as |
| | | DTT or TCEP. |
| 4 | Huisgen 1,3 di-polar | Typically Cu or Ru catalyzed |
| | cycloaddition. | reaction between azide and |
| | | alkyene, but can include |
| | | electron deficient nitriles. |
| 5 | Amide from corridponding | Standard peptide coupling |
| | amine and carboxylic acid | conditions eg coupling |
| | | reagent HATU, HOBt, COMU |
| | | with base. |
| 6 | Singlet or triplet based | UV irridation at ≈ 355 nm |
| | carbene reaction | |
| 7 | Olefin metathesis | Ruthenium based grubbs |
| | | generation 1 and 2 and |
| | | Grubbs-Hoyveda gen 1 and 2 |
| 8 | Suziki or Sonogashira | Palladium based catalysts |
| | coupling | |

All the peptide components (amino acids, unnatural amino acids, unstapled/unstitched, partially stapled/stitched and stapled/stitched peptides) may exist in specific geometric or stereoisomeric forms. All compounds include cis- and trans-isomers, (R)- and (S)-enantiomers, diastereoisomers and racemic mixtures thereof.

Preferred isomer/enantiomers will be enriched to give a greater proportion of one particular isomer or enantiomer. Embodiments thereof may be made of greater than 90%, 95%, 98% or 99%, by weight, of a preferred isomer/enantiomer.

Non-limiting examples of unnatural amino acids used in stabilising a peptide structure are illustrated in Table 1.

In PCT/GB2016/054028 the applicant employed α,α-disubstituted unnatural amino acids bearing all-hydrocarbon tethers (e.g. α-methyl, α-pentenyl glycine).

In the present invention the applicant in the alternative employs the chemistries disclosed in Table 6.

In one preferred embodiment they form a cross link by coupling two naturally occurring amino acid (e.g. lysine and glutamic acid) in the sequence RKF-[E-RLF-K]. Alternatively, these naturally occurring amino acids could be lysine and aspartic acid.

In yet another embodiment the applicant employs a cross link between a modified natural amino acid (N⁶-diazolysine) and a non-natural amino acid (S)-2-Amino-2-methyl-4-pentynoic acid such as the sequence RKF—[K(N₃)—RLF-B] where B represents (S)-2-Amino-2-methyl-4-pentynoic acid and K(N₃) represent N⁶-diazolysine.

In one embodiment the cell penetrating agent has a staple or stitch peptide comprising the sequence RFK—X-RLF-X, where X represents an amino acid that is able to be cross linked.

In another embodiment the sequence RFK—[X-RLF-X] could have the relative position of the cross linking residues moved, for example, but not limited to RF—[X—KRLF-X] or RFKR—[X-LF-X].

In another embodiment the peptide is a branched stapled peptide. The branched stapled peptide comprises of 2 or more chains of peptides. Branched peptides may be formed using any method know to the art; in one embodiment a lysine residue is used to branch two peptide chains.

Functional derivatives of disclosed peptide sequences could be used. Functional derivatives may have representative fragments or homologues or peptides that include insertions to the original peptide. Typical derivative would have 70%, 80%, 90% or more of the original peptide sequence and may have up to 200% of the number of amino acids of the original peptide. The derivatives would be used to enhance the delivery of a biologically active compound.

Peptide sequence can include modified amino acids (Table 1) to include functional groups that permit the addition of other moieties. Non-limiting examples of such moieties include an acetyl, a cholesterol, a fatty acid, a polyethylene glycol, a polysaccharide, an aminoglycan, a glycolipid, a phospholipid, a polyphenol, a nuclear localising signal, a nuclear export signal, an antibody and a targeting molecule.

### 3. Bi-Functional Linker

A bi-functional linker may be used to link the BAC to the CPA.

Preferred linkers will link between, for example, an amine group on the BAC and a sulfhydryl (thiol) group (usually a cysteine residue) on the CPA terminus. Examples of substrates to achieve this include, but are not limited to, SMCC (succinimidyl 4-(N-maleimidomethyl)cyclohexane-1-carboxylate), AMAS (N-α-maleimidoacet-oxysuccinimide ester, BMPS (N-β-maleimidopropyl-oxysuccinimide ester), GMBS (N-γ-aleimidobutyryl-oxysuccinimide ester), DMVS (N-□-maleimidovaleryl-oxysuccinimide ester, EMCS (N-ε-maleimidocaproyl-oxysuccinimide ester), and LC-SMCC (Succinimidyl 4-(N-maleimidomethyl)cyclohexane-1-carboxy-(6-amidocaproate) as exemplified in Table 5.

Another preferred linker system is hydrazynal nicotinic acid (HNA), however if the BAC is a PMO, the PMO is modified to incorporate 4 formyl benzoic acid.

Other linkers such as DSG (disuccinimidyl glutarate) and DSCDS (disuccinimidyl-cyclohexl-1,4-diester) will include the ability to link the 5′-amino group of the BAC to the N-terminus of the CPA (Table 5, entries 9 and 10).

Linkers may include other elements that confer a desirable property on the DCCPM e.g. spacer between ON and CPA or an element that will enhance solubility, for example a PEGylated element. Non-limiting examples are shown in Table 5.

The biologically active compound is covalently attached to the chimeric cell delivery peptide. Again, this can be done using any method known in the art. Preferably, the cell delivery peptide is attached to the biologically active compound by means of a disulphide bridge or a thiol maleimide linker e.g. SMCC; the attachment may be by means of an amide linker or an oxime linker or a thioether linker.

### EXAMPLES

### Proof of Principal Provided in PCT/GB2016/054028 with PMO CP8M and PMO HP8M which Serve as Comparators to the Alternative Chemistries Described Herein

DCCPM to enhance RNA steric blocking in treating Duchenne muscular dystrophy (DMD)

### Introduction

Duchenne muscular dystrophy (DMD) is the most common inherited lethal childhood disease in the world, with a worldwide incidence of approximately 1 in 4000 live births³⁷. This severe muscle-wasting disorder is caused in the majority of families by gene mutations leading to disruption of the reading frame and premature truncation of the protein dystrophin^{38,39}.

RNA splicing suppression of the DMD transcript has particular promise. The hybridisation of AOs to specific RNA sequence motifs prevents correct assembly of the spliceosome, so that it is unable to recognise the target exon(s) in the pre-mRNA and hence excludes them in the mature gene transcript. AO-mediated RNA splicing suppression resulting in the re-expression of a truncated, yet functional dystrophin protein has been demonstrated in vitro and in the pre-clinical mdx mouse model^{32,40-45}, which led to clinical development programs^{2,10}.

Although intravenously administered PMOs have demonstrated a dose-dependent increase in dystrophin re-expression with some functional benefit^{2,46}, skeletal muscle dystrophin restoration is still very variable between patients after many multiple administrations. Importantly, many other target tissues (e.g. brain and heart) remain refractory to PMO transfection even when repeat administration or high dose strategies are employed^{30,32}.

To date unmodified CPA conjugation improves PMO bio-distribution and serum stability³³⁻³⁵, however toxicity is still a major roadblock for pipeline development²⁰.

The applicant hypothesised that a CPA based upon a stabilized e.g. StaP (or StiP) conjugated to a PMO known to cause RNA splicing suppression of the DMD transcript, would lead to a greater level of dystrophin restoration and re-expression of dystrophin in tissues refractory to naked PMO without the potential for CPA related toxicity. We bring data forward that demonstrate novel CPA have attractive biological and toxicological properties, such that these novel DCCPMs or DTCPMs are clinically relevant.

### Materials and Methods

### General Peptide Synthesis Procedure

For ring closing metathesis peptides, all peptides were synthesized following an established protocol using standard Fmoc-peptide chemistry on Rink amide MBHA resin. The coupling reactions were performed by the addition of a mixture of 10 equivalents of the amino acids, 9.9 equivalents of HCTU and 20 equivalents of DIPEA in NMP (equivalents relative to initial loading of Rink amide MBHA resin). The reactions were allowed to proceed for at least one hour. Coupling of non-natural amino acids (R/S5, R/S8 or B5) was performed with 4 equivalents of the amino acid, 3.9 equivalents of HCTU and 10 equivalents of DIPEA in NMP for two hours. The ring closing metathesis reaction of the olefin-containing non-natural amino acids was facilitated with Grubbs I catalyst (benzylidene-bis(tricyclohexylphosphine)-dichlororuthenium) dissolved to approximately 10 mg/mL in 1,2-dichloroethane (DCE) for two hours under nitrogen bubbling. Subsequently, excess catalyst was washed from the resin with DCE and then coupled with an N-terminal FITC. Upon completion, peptides were simultaneously cleaved from the resin and de-protected using a cleavage cocktail containing 95% TFA, 2.5% TIS and 2.5% water. Crude peptides were dissolved in 50% acetonitrile/water, passed through a 0.2 μm syringe filter, and purified by reverse phase HPLC using a C-18 column (Agilent, Palo Alto, Calif.). Compound identification and purity was assessed using coupled LC/MS (Agilent, Palo Alto, Calif.). Purified fractions were pooled and evaporated to remove acetonitrile and trace TFA by Speedvac and then lyophilized to dryness. A non-ring closed peptide was also produced as a control.

All peptides were synthesized following an established protocol using standard Fmoc-peptide chemistry on Rink amide MBHA resin or 2-chlorotrytyl resin for the free acid variants. The coupling reactions were performed by the addition of a mixture of 10 equivalents of the amino acids, 9.9 equivalents of HCTU and 20 equivalents of DIPEA in NMP (equivalents relative to initial loading of Rink amide MBHA resin). The reactions were allowed to proceed for at least one hour.

### Synthesis of Peptide Containing Modified Amino Acids and Un-Natural Amino Acids.

Deprotection of the temporary Fmoc group was achieved by 2×20 min treatments of the resin-bound peptide with 20% (v/v) piperidine in DMF. After extensive flow washing with DMF, coupling of each successive amino acid was achieved with 1×30 min incubation with the appropriate preactivated NR-Fmoc-amino acid derivative. All protected amino acids (1 mmol) were dissolved in the cartridge with 3.8 mL of 0.25 M DEPBT in DMF as part of the synthesizer program immediately before delivery to the reaction vessel. Subsequently, 1 mL of DIEA was added directly to the cartridge to effect activation no more than two minutes prior to transfer of the coupling solution to the NR-deprotected resin-bound peptide. After coupling was complete, the resin was extensively flow washed in preparation for the next deprotection/coupling cycle.

Synthesis for Lactam Containing Amino Acids (e.g. K1E5)

Side-chain protection of glutamic acid and lysine consisted of Fm and Fmoc groups, respectively. After acetylation amino terminus with Ac₂O/NMM (2 mmol each, 114 and 189 μL, respectively) in a manner similar to any other NR-Boc-amino acid, Fmoc, and Fm side-chain protecting groups were removed with 20% piperidine, the resin-bound peptide was washed with DMF and then treated for 2 h with 1.9 mmol of HBTU (3.8 mL×0.5 M in DMF) and 1.9 mmol of DIEA to effect the lactam formation.

Selective Transformation of Lysine to N⁶-diazolysine

The target lysine residue for transformation was protected with an Mtt protecting group where as other lysine residues that do not require transformation are protected with Boc protecting group. The resin-bound peptide (0.31 mmol/g, 300 mg) in this instance the sequence RK(Boc)F—K(Mtt)-RLF-B, where B is the incorporated non-natural amino acid (S)—N-Fmoc-2-(2′-propynyl)alanine was suspended in 1% TFA solution in DCM (10 mL) and was stirred for 3 min. The solution becomes yellow instantaneously. Then the resin was washed with DCM (2×), MeOH (1×), and DCM (2×). The process was repeated 8 times until the solution stayed colorless. The resin-bound peptide was taken to the next step without further manipulations. Triflic acid anhydride (Tf₂O, 316 μL, 1.87 mmol) was added dropwise to a vigorously stirred mixture of NaN3 (600 mg, 9.2 mmol) in H₂O (1.5 mL) and CH₂Cl₂ (3 mL) at 0° C. The resulting mixture was allowed to warm to room temperature and was stirred for 2 h. The water layer was extracted twice with CH₂Cl₂, and the combined organic layers were washed with saturated aqueous Na₂CO₃. The resulting solution of TfN₃ in CH₂Cl₂ was added slowly to the resin-bound peptide suspended in a solution of CuSO₄.5H₂O (2 mg, 8 mmol) and K₂CO₃ (5 mg, 36 mmol) in MeOH (1 mL). This reaction mixture was swirled for 18 h at room temperature. The completeness of the diazo transfer could be followed with the Kaiser test; colorless resin beads implied that the conversion of the amino group into the azido functionality had been completed. The resin was subsequently washed sequentially with DCM, MeOH, DMF, and DCM.

### Cleavage and De Protection of the Peptide

The azide/alkynyl-containing resin-bound peptide was deprotected and cleaved from the solid support by treatment with TFA/TIS/H₂O (95:2.5:2.5 v/v) for 4 h at RT. After filtration of the resin, the TFA solution was concentrated under reduced pressure and precipitated in ether to yield the desired product as a solid which was then purified by reverse phase chromatography.

### High Resolution Mass Spectroscopy

High-resolution mass spectra were recorded on a Thermo scientific LQT Orbitrap XL under electron spray ionization conditions (ESI) or where indicated under Atmospheric Pressure Ionisation (API) condition.

### Circular Dichroism (CD) Spectroscopy

CD analysis was performed on an Applied Photophysics Chirascan Circular Dichroism spectrometer. Samples were dissolved in D₂O at a maximum of 0.125 W/W % and data acquired in triplicate at room temperature and subsequently averaged and smoothed using built in qCD software. Graphs were plotted by subtracting a blank D₂O spectrum from the acquired data to provided blank correction.

### Synthesis of PMO-CP8M

PMO (91.64 mg, 10 μmol) was dissolved in PBS (5 mL, pH 7.2) and incubated at room temp after the addition of SMCC linker (27.2 mg, 50 μM, 5× excess) dissolved in MeCN/H₂O (1:1, 1 mL). After 30 mins the mixture was desalted using sephadex g25 hydrated in conjugation buffer (PBS 1×, pH 6.8) and was also used as the eluent.

K1E4-CP8M (17.8 mg, 11.4 μmol) was dissolved in milliQ water (4 mL) and EDTA solution (0.1 mL, 100 mM) and mixed with immobilised TCEP (2.5 mL) for 1 h prior. Final concentration of EDTA was 2 mM.

The freshly desalted SMCC modified PMO had MeCN (8 mL) and EDTA solution (0.1 mL, 100 mM) added before adding peptide the addition of the peptide. The reduced peptide was eluted from the immobilised TCEP into a tube containing the SMCC modified PMO and stirred at RT for 2 hours.

The solution was loaded on to 3×560 mg HLB columns, and washed with milliQ water to remove any salts, then 10% MeCN in water. When washing with 20% MeCN an amount of PMO was removed from the column. 20% MeCN was sufficient to remove unconjugated PMO from the HLB columns. Columns were washed with 20% MeCN until the eluent ran clear. Finally PMO conjugate was eluted with 50% MeCN in water. The eluted products then underwent size exclusion chromatography using sephadex superfine g25 hydrated in milliQ water also used as the eluent.

### Synthesis of PMO-HP8M and Modification of PMO to PMO-4FB.

4-FB (250 mg, 1.5 mM) was dissolved in DMF with COMU (1.2 g, 2.6 mM) and NHS (230 mg, 2.0 mM) and stirred for a few mins. 4-FB did not fully dissolve until DIEA was added. DIEA (0.54 mL 3.0 mM) was then added upon which the reaction mixture changed from colourless to pale yellow/orange. The reaction mixture was stirred for 1 h and monitored by TLC using 5% MeOH in DCM. The mixture was separated over DCM to remove DMF then purified by flash chromatography using DMC to elute the top spot staining positive with 2,4 DNP. Product was collected as an off white solid 112 mg (30%).

PMO (30.4 mg, 3 μM) was added to a solution of 4-FB and dissolved in 10 Carbonate buffer:MeCN (50% MeCN) and NHS activated 4-FB (10 mg, 32 μM) was added and stirred overnight. The mixture was then desalted using sephadex G25 superfine with water:MeCN as an eluent. MeCN was removed by rotary evaporation and the remaining eluent was then freeze dried. Freeze dried product yielded 24 mg 83% yield.

### Conjugation of PMO-4FB to HP8M

HP8M was dissolved in milliQ ultra-pure water (100 μL) to give a solution of 12 mg/mL. Aldehyde modified PMO (7 mg, 0.76 μM) was dissolved in water/MeCN (300 μL, 1:1) and desalted using sephadex G25 superfine and water/MeCN (1:1) as the eluent. The collected fraction was then diluted to 1 mL total volume in water:MeCN mix (1:1) and PMO content was analysed by UV/vis and found to be 6.5 mg/mL or 705 μM. HNA peptide and Analine (10 mM final conc) was then added and UV/vis monitored for evidence of A₃₅₄ and used to calculate the conjugation of PMO to peptide.

### Cell Culture and Transfection

HeLa pLuc705 cells were cultured in high glucose DMEM supplemented with 10% foetal calf serum (Sigma, UK) at 37° C. under an 8% CO₂/92% air atmosphere.

HeLa pLuc705 cells were setup in 96 well plates with the appropriate dilutions of test compounds either FITC labeled peptides or FITC labeled PMO conjugates diluted into complete culture media (up to 100□M). Cells were then then trypsinised, diluted to 4×10⁵ cells per mL and 100 μL added to each well giving a final volume of 200 μL in each well. Cells were then incubated for either 4 or 24 hours at either 4 or 37° c.

### Flow Cytometry

Uptake of fluorescently-labelled PMO and peptides was determined by flow cytometry using an Accuri C6 flow cytometer. Cells were washed with PBS and glycine buffer then released with trypsin, and kept on ice before analysis in PBS containing 2.5% FBS. Cell fluorescence in single live cells was determined using FlowJo software after appropriate gating. Untreated cells were used to establish gating settings for the determination of the % fluorescein-positive cells, mean fluorescent intensity (MFI) was also calculated. Uptake was determined by gating cells that were able exclude cell-impermanent die (To-pro-3) indicating the ability of cells to retain membrane integrity.

### HPLC Analysis

Samples were run on a Kinetex, 2.6 μM particles size, XB—C18 modified with 100 Å pores. Samples were run on a gradient of 0-80% MeCN over 8 mins at a flow rate of 1.5 mL/min at 60° C.

### Statistical Analysis

All data are reported as mean values±SEM or standard deviation as indicated. Statistical differences between treatment groups and control groups were evaluated by SigmaStat (Systat Software, UK) and student's t test was applied or 2 way ANOVA. Significance was accepted for p-values<0.05 using a bonferroni post hoc analysis.

### Results

Solid phase synthesis of stabilised peptides K1E4/5/6-P8M-FITC and the non-cyclised equivalents K1E4/5/6-P8M-NC—FITC, RCM1,5-P8M-FITC and equivalent non cyclised RCM1,5-P8M-FITC-NC, RCM1,5-CP8M and equivalent non cyclised RCM-CP8M-NC and RCM1,5-HP8M was performed with standard Fmoc chemistry on Rink amide resin and yielded the desired product identified by LC-MS in Table 7 below.

TABLE 7

| | | Calculated | Observed |
|---|---|---|---|
| | | Molecular | Molecular |
| Sample | Formula | mass (Av) | Mass (Av) |
| K1E4-P8M-FITC | C₈₉H₁₂₅N₁₉O₂₁S | 1829.1510 | 1829.353 |
| K1E4-FITC-NC | C₈₉H₁₂₇N₁₉O₂₂S | 1847.1660 | 1847.353 |
| K1E5-FITC | C₈₉H₁₂₅N₁₉O₂₁S | 1829.1510 | 1829.353 |
| K1E5-FITC-NC | C₈₉H₁₂₇N₁₉O₂₂S | 1847.1660 | 1847.353 |
| K1E6-FITC | C₈₉H₁₂₅N₁₉O₂₁S | 1829.1510 | 1829.353 |
| K1E6-FITC-NC | C₈₉H₁₂₇N₁₉O₂₂S | 1847.1660 | 1847.353 |
| RCM1,5-P8M-FITC | C₉₂H₁₃₀N₁₈O₂₀S | 1840.2180 | 1840.387 |
| RCM1,5-P8M-NC-FITC | C₉₄H₁₃₄N₁₈O₂₀S | 1868.2720 | 1868.387 |
| RCM1,5-CP8M | C₇₄H₁₂₄N₁₈O₁₆S | 1553.9760 | 1554.150 |

HPLC analysis of un-cyclised peptides K1E4/5/6-CP8M-FITC-NC showed elution time of 6.22-6.29 mins using a 0-80% gradient over 8 mins.

HPLC analysis of cyclised peptides K1E4/5/6-CP8M-FITC showed in increase in elution time over the un-cyclised peptide (Table 8).

HPLC analysis of RCM1,5-CP8M showed a decrease in retention time of the cyclised product compared to un-cyclised product with a similar retention time to K1E4-CP8M and K1E6-CP8M peptides as illustrated in Table 8 below.

TABLE 8

| Peptide | Not-Cyclised retention time | Cyclised retention time |
|---|---|---|
| K1E4 | 6.295 | 6.625 |
| K1E5 | 6.284 | 6.820 |
| K1E6 | 6.225 | 6.571 |
| RCM1,5 | 6.858 | 6.723 |

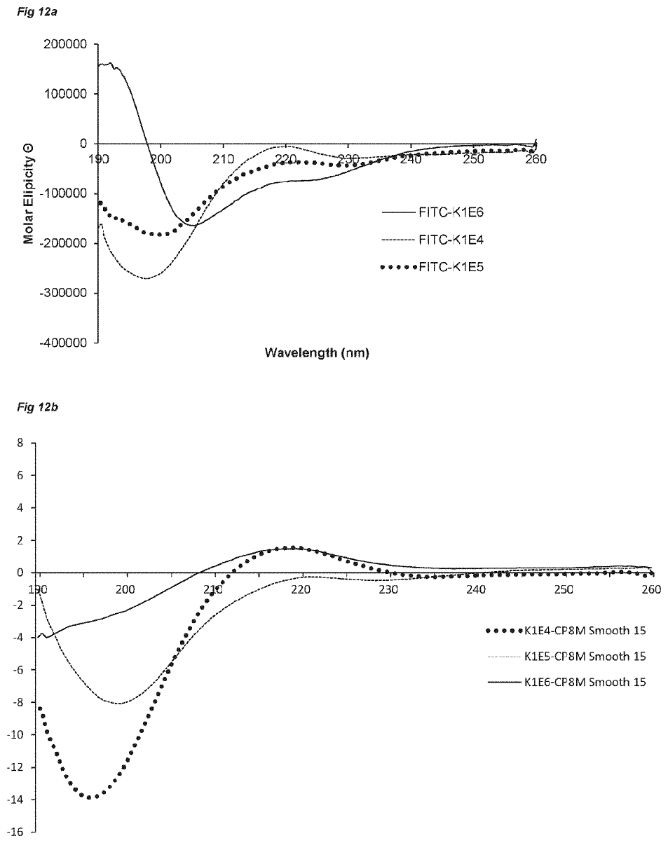

Circular dichroism data indicates the solvated structure of K1E4/5/6-CP8M peptides can be influenced by both the presence of a charged fluorochrome or the position of the cross-link (FIGS. 12a and 12b).

K1E4-CP8M exhibits an extended 3₁₀Helix/Poly (pro)II helix with maxima at 219 nm and minima of 196 nm. K1E6-CP8M show similar characteristic maxima and minima (FIG. 12).

K1E5-CP8M displays characteristics of random coil or disordered structure (FIG. 12) displayed by the low near 8 elipicity above 210 nm and a minima around 196 nm.

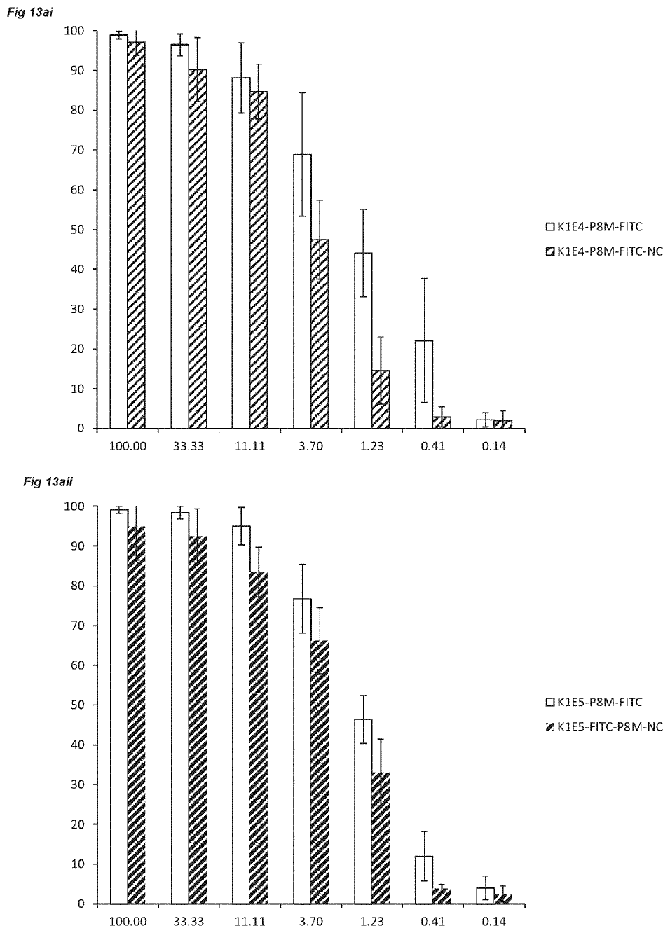

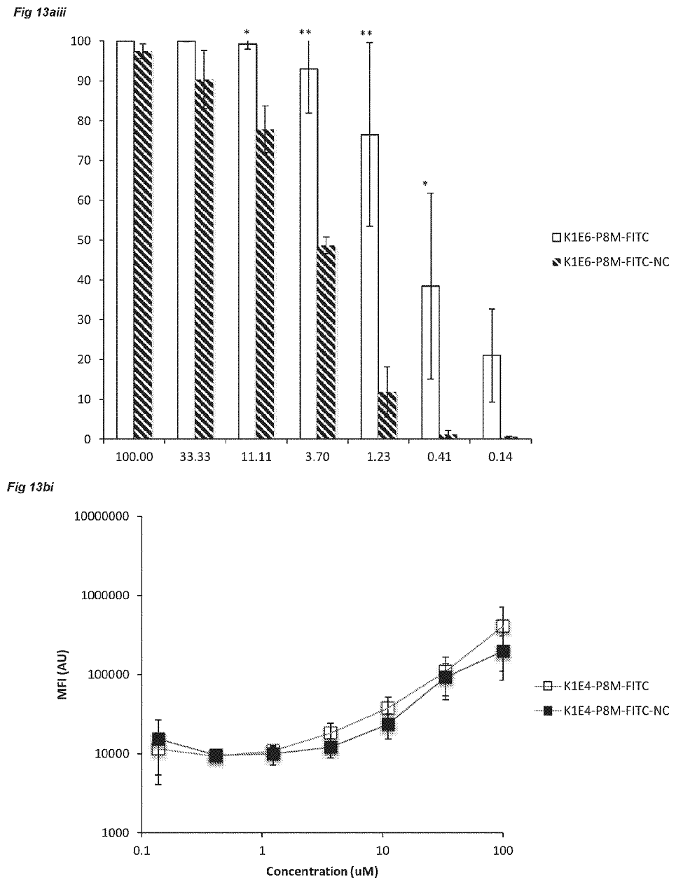

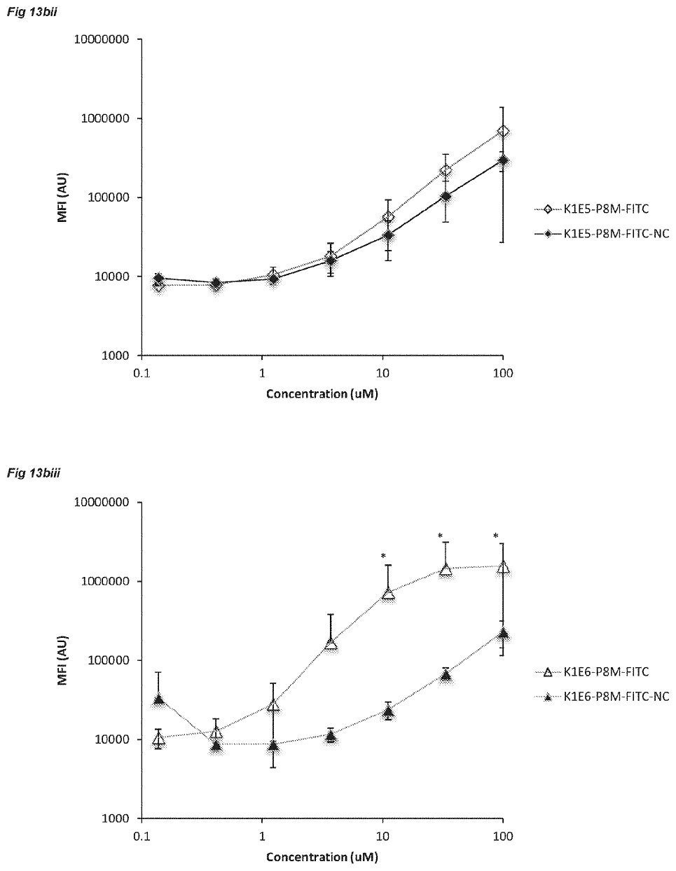

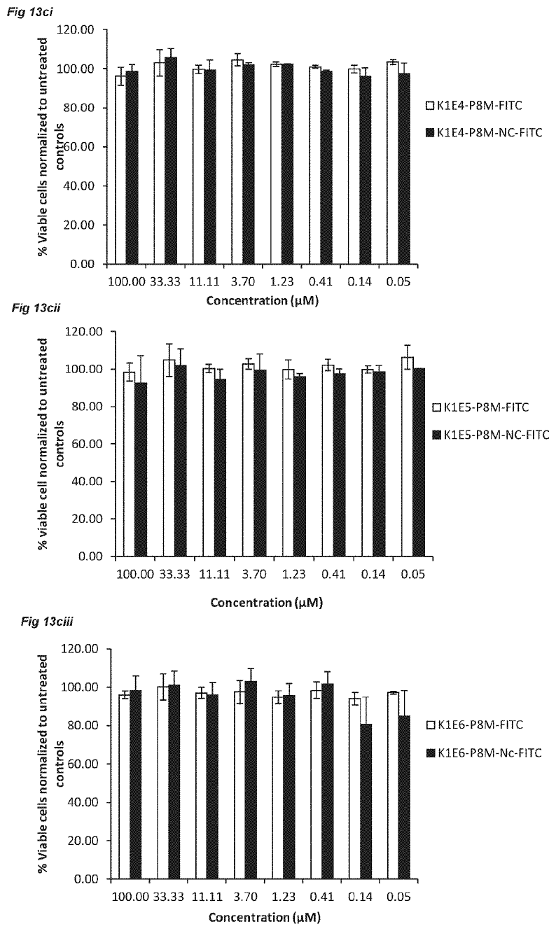

HeLa pLuc 705 cells incubated in the presence of K1E4/5-CP8M-FITC and K1E4/5/6-CP8M-NC—FITC showed no difference in the uptake of stabilised peptides vs non-stabilised peptides (FIG. 13ai-aii). Surprisingly, K1E6-CP8M did show a significant difference in uptake compared to the non-stabilised peptide (FIG. 13aiii; n=4, error bars show standard deviation 2 way ANOVA was performed * represents p<0.05, ** represents p<0.001). A 22% increase in FL1+ cells was observed at 11.1 μM when comparing K1E6-CP8M-FITC to the uncyclised control. The difference in FL1+ cells further increased to 43% difference at 3.7 μM and at the largest difference of 65% at 1.23 μM and remained constantly higher at all tested concentration. This demonstrates that the position of the cross link that influences cellular uptake is not intuitive between different cyclisation technologies as PCT/GB2016/054028 demonstrates that ring closing metathesis between amino acids a position 1 and 5 within a peptide are efficient at cellular entry.

HeLa pLuc 705 cells incubated in the presence of K1E4/5-CP8M-FITC and K1E4/5/6-CP8M-NC—FITC demonstrated that a significant increase of over 2 logs in mean fluorescent intensity was only observed for K1E6-CP8M-FITC peptide (compared to it non cyclised control) at concentration greater than 10 μM; FIG. 13bi-biii; n=4, error bars show standard deviation. 2 way ANOVA was performed * represents p<0.05).

HeLa pLuc 705 cells incubated in the presence of K1E4/5-CP8M-FITC and K1E4/5/6-CP8M-NC—FITC demonstrate no adverse cellular toxicity across all concentration ranges (0.05□M to 100□M; FIG. 13ci-iii).

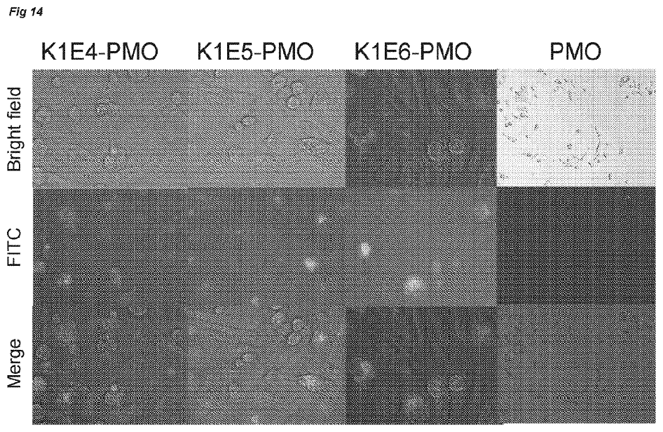

HeLa pLuc 705 cells incubated in the presence of K1E4/5/6-CP8M-conjugated PMO demonstrated an increase in intracellular uptake compared to unconjugated PMO (FIG. 14).

Hela pLuc 705 cells incubated in the presence of RCM1,5-CP8M-FITC labelled peptide shows a dose dependent uptake of peptide at similar levels to K1E6-CP8M-FITC peptide.

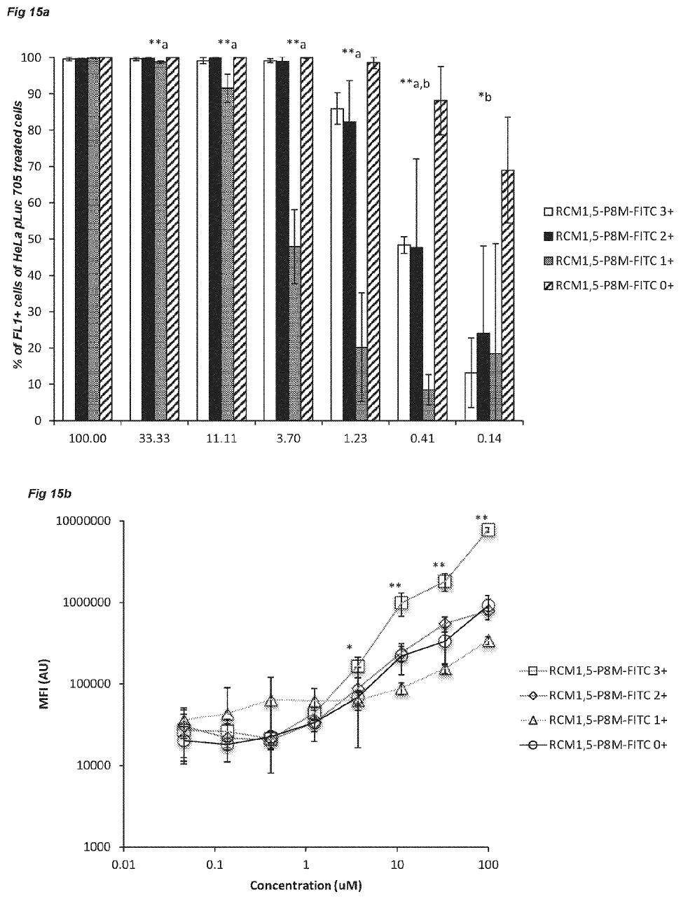

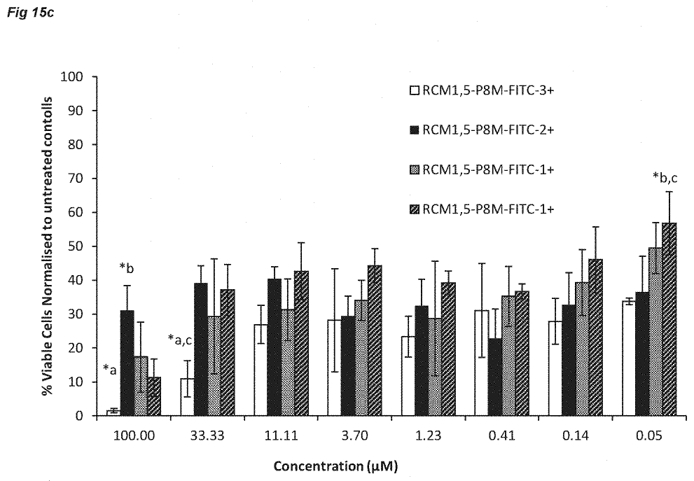

Hela pLuc 705 cells incubated in the presence of FITC labelled peptides based on charge variants of RCM1,5-CP8M-FITC (FIG. 14a; i.e. RCM1,5-CP8M-FITC 3+, RCM1,5-CP8M-FITC 2+, RCM1,5-CP8M-FITC 1+ and RCM1,5-CP8M-FITC 0+). FIG. 15a demonstrated a differential response to peptide dose and cell viability. Data shows that RCM1,5-P8M-FITC-1+ has a significant reduction in FL1+ cells at all concentration below 33 μM compared to other treatments, ranging from 2% difference to 60% difference of FL1 positive cells. RCM1,5-P8M-FITC-0+ shows in increase in the number of FL1+ cells over a range of concentrations compared to other treatments ranging from a 10% increase to 40% increase at the largest difference at 0.41 μM (n=5, error bars show standard deviation. * represents p<0.05, ** represents p<0.001, *a represents an interaction between RCM1,5-P8M-FITC-1+ and all other groups, *b represents an interaction between RCM1,5-P8M-FITC-0+ and all other groups).

Hela pLuc 705 cells incubated in the presence of FITC labelled peptides based on charge variants of RCM1,5-CP8M-FITC had differential mean fluorescent intensities (FIG. 15b). There is no significant difference in the MFI between RCM1,5-P8M-FITC-0+, 1+ and 2+ at a given concentration. However RCM1,5-P8M-FITC-3+ shows an increase in MFI at higher concentrations >1 μM maximally about 1 log increase (n=5, error bars show standard deviation. * represents p<0.05, ** represents p<0.001).

FIG. 15c is a comparison of % viable cells judged by gated cells compared to negative controls of HeLa pLuc 705 cells incubated with RCM1,5-P8M-FITC-3+, RCM1,5-P8M-FITC-2+, RCM1,5-P8M-FITC-1+, RCM1,5-P8M-FITC-0+ for 4 hours (n=3, error bars show standard deviation * represents p<0.05, *a represents an interaction between RCM1,5-P8M-FITC-3+ and RCM1,5-P8M-FITC-2+, *b represents an interaction between RCM1,5-P8M-FITC-2+ and RCM1,5-P8M-FITC-0+ and *c represents an interaction between RCM1,5-P8M-FITC-0+ and RCM1,5-P8M-FITC-3+). Data shows that at highest concentration 100 μM a significant loss in the number of viable cells recovered when comparing RCM1,5-CP8M-FITC-3+ to RCM1,5-CP8M-FITC-2+(32% reduction) and an increase in the number of cells when comparing RCM1,5-CP8M-FITC-2+ to RCM1,5-CP8M-FITC-0+(20% increase). Similarly at 33 μM RCM1,5-CP8M-FITC-3+ compared to RCM1,5-CP8M-FITC-2+ and RCM1,5-CP8M-FITC-0+ showed a 20% decrease in the % viable cells. At all other concentration no other significant affects were observed apart from at 0.05 μM. At 0.05 μM RCM1,5-CP8M-FITC-0+ showed an increase of 23% in viable cells over RCM1,5-CP8M-FITC-3+ and RCM1,5-CP8M-FITC-2+. This has important implications for clinical translation of reduce charge RCM based CPP variants.

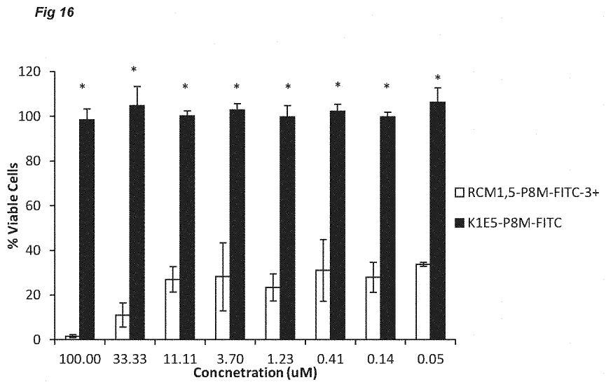

Comparisons of HeLa pLuc 705 cell viability when incubated with either RCM1,5-P8M-FITC-3+ or any of the K1E4/5/6-CP8M-FITC series of peptide demonstrates that at dose ranges 0.05 □100 □M, that the K1E4/5/6-CP8M-FITC do not have any negative impact on cell viability (FIG. 16). Comparing RCM1,5-P8M-FITC to K1E5-P8M-FITC peptides show an increase in viability of 70-99% over the range of concentrations. This has important implications for clinical translation of lactamisation based CPPs.

### CONCLUSION

From the data generated it can be seen that a CPA stabilized by stapling via alternate cross linking technologies to that the applicant disclosed in PCT/GB2016/054028 are effective at cell entry in vitro.

The importance of position of the crosslink within the peptide sequence has been illustrated as chemistry specific and can greatly influence the solvated conformation of the stabilised peptide and subsequently the cellular uptake as measured by flow cytometry. Thus, surprisingly, it is not intuitive that crosslinks based on different chemical cyclisation technologies, orthogonal to the sequence, generate peptides with either the same conformation or the same cell entry properties. This may be true for other cyclisation chemistries.

CPAs stabilized by lactamisation cyclisation chemistry stabilize into helical structures and the structures are not □-helical. K1E4-CP8M and K1E6-CP8M exhibits an extended 3₁₀Helix/Poly (pro)II helix structure.

CPAs stabilized by lactamisation cyclisation chemistry does not cause cellular death in vitro. This has important clinical translation implication for DCCPMs based on this technology.

HPLC analysis of cyclised and non-cyclised K1E4/5/6-CP8M peptides illustrated the similar retention time of non-cyclised peptide 6.22 min (Table 8), however the cyclisation process and resulting stabilised peptides showed an increase in retention times (Table 8). Peptides with inferred conformation of an 3₁₀Helix/Poly (pro)II helix had broadly similar retention times K1E4-CP8M=6.62 and K1E6-CP8M=6.57 mins highlighting the potential use of HPLC for identifying changes in conformation.

CPAs stabilized by lactamisation cyclisation chemistry when conjugated to a PMO facilitates the cellular entry of the PMO.

Reduced charge variants of CPAs stabilized by a ring closing metathesis cyclisation chemistry are efficient cell entry peptides and have improved toxicological profiles in vitro. This has important clinical translation implication for DCCPMs based on this technology.

### REFERENCES

1 Chiriboga, C. A. et al. Results from a phase 1 study of nusinersen (ISIS-SMN(Rx)) in children with spinal muscular atrophy. Neurology 86, 890-897, doi:10.1212/WNL.0000000000002445 (2016).
2 Mendell, J. R. et al. Eteplirsen for the treatment of Duchenne muscular dystrophy. Ann Neurol 74, 637-647, doi:10.1002/ana.23982 (2013).
3 Iversen, P. L. et al. Discovery and early development of AVI-7537 and AVI-7288 for the treatment of Ebola virus and Marburg virus infections. Viruses 4, 2806-2830, doi:10.3390/v4112806 (2012).
4 Heald, A. E. et al. Safety and pharmacokinetic profiles of phosphorodiamidate morpholino oligomers with activity against ebola virus and marburg virus: results of two single-ascending-dose studies. Antimicrob Agents Chemother 58, 6639-6647, doi:10.1128/aac.03442-14 (2014).
5 Warren, T. K. et al. Advanced antisense therapies for postexposure protection against lethal filovirus infections. Nat Med 16, 991-994, doi:10.1038/nm.2202 (2010).
6 Campbell, J. M., Bacon, T. A. & Wickstrom, E. Oligodeoxynucleoside phosphorothioate stability in subcellular extracts, culture media, sera and cerebrospinal fluid. Journal of biochemical and biophysical methods 20, 259-267 (1990).
7 Agrawal, S., Mayrand, S. H., Zamecnik, P. C. & Pederson, T. Site-specific excision from RNA by RNase H and mixed-phosphate-backbone oligodeoxynucleotides. Proc Natl Acad Sci USA 87, 1401-1405 (1990).
8 Tereshko, V. et al. Correlating structure and stability of DNA duplexes with incorporated 2′-O-modified RNA analogues. Biochemistry 37, 10626-10634, doi:10.1021/bi980392a (1998).
9 Shibahara, S. et al. Inhibition of human immunodeficiency virus (HIV-1) replication by synthetic oligo-RNA derivatives. Nucleic Acids Res 17, 239-252 (1989).
10 Goemans N, C. C., Kraus J E, et al. Drisapersen efficacy and safety in Duchenne muscular dystrophy: results of a phase III, randomized, double-blind, placebo-controlled trial (study DMD114044). World Muscle Society Congress; Asilomar, Calif., USA; Oct. 1-5, 2013 (2103).
11 Goemans, N. M. et al. Long-Term Efficacy, Safety, and Pharmacokinetics of Drisapersen in Duchenne Muscular Dystrophy: Results from an Open-Label Extension Study. PLoS One 11, e0161955, doi:10.1371/journal.pone.0161955 (2016).
12 Dirin, M. & Winkler, J. Influence of diverse chemical modifications on the ADME characteristics and toxicology of antisense oligonucleotides. Expert Opin Biol Ther 13, 875-888, doi:10.1517/14712598.2013.774366 (2013).
13 Sazani, P. et al. Repeat-dose toxicology evaluation in cynomolgus monkeys of AVI-4658, a phosphorodiamidate morpholino oligomer (PMO) drug for the treatment of duchenne muscular dystrophy. Int J Toxicol 30, 313-321, doi:10.1177/1091581811403505 (2011).
14 Sazani, P., Weller, D. L. & Shrewsbury, S. B. Safety pharmacology and genotoxicity evaluation of AVI-4658. Int J Toxicol 29, 143-156, doi:10.1177/1091581809359206 (2010).
15 Heemskerk, H. A. et al. In vivo comparison of 2′-O-methyl phosphorothioate and morpholino antisense oligonucleotides for Duchenne muscular dystrophy exon skipping. J Gene Med 11, 257-266, doi:10.1002/jgm.1288 [doi] (2009).
16 Kreutz, M. et al. Antibody-antigen-adjuvant conjugates enable co-delivery of antigen and adjuvant to dendritic cells in cis but only have partial targeting specificity. PLoS One 7, e40208, doi:10.1371/journal.pone.0040208 (2012).
17 Derossi, D., Joliot, A. H., Chassaing, G. & Prochiantz, A. The third helix of the Antennapedia homeodomain translocates through biological membranes. J Biol Chem 269, 10444-10450 (1994).
18 Gautam, A. et al. CPPsite: a curated database of cell penetrating peptides. Database the journal of biological databases and curation 2012, bas015, doi:10.1093/database/bas015 (2012).
19 Hirose, H. et al. Transient focal membrane deformation induced by arginine-rich peptides leads to their direct penetration into cells. Mol Ther 20, 984-993, doi:10.1038/mt.2011.313 (2012).
20 Moulton, H. M. & Moulton, J. D. Morpholinos and their peptide conjugates: therapeutic promise and challenge for Duchenne muscular dystrophy. Biochim Biophys Acta 1798, 2296-2303, doi:10.1016/j.bbamem.2010.02.012 (2010).
21 Tunnemann, G. et al. Live-cell analysis of cell penetration ability and toxicity of oligo-arginines. J Pept Sci 14, 469-476, doi:10.1002/psc.968 (2008).
22 Chu, Q. et al. Towards understanding cell penetration by stapled peptides. MedChemComm 6, 111-119, doi:10.1039/C4MD00131A (2015).
23 Hilinski, G. J. et al. Stitched α-Helical Peptides via Bis Ring-Closing Metathesis. Journal of the American Chemical Society 136, 12314-12322, doi:10.1021/ja505141j (2014).
24 Cantel, S. et al. Synthesis and conformational analysis of a cyclic peptide obtained via i to i+4 intramolecular side-chain to side-chain azide-alkyne 1,3-dipolar cycloaddition. The Journal of organic chemistry 73, 5663-5674, doi:10.1021/jo800142s (2008).
25 Bracken, C., Gulyas, J., Taylor, J. W. & Baum, J. Synthesis and Nuclear Magnetic Resonance Structure Determination of an .alpha.-Helical, Bicyclic, Lactam-Bridged Hexapeptide. Journal of the American Chemical Society 116, 6431-6432, doi:10.1021/ja00093a052 (1994).
26 Lehto, T. et al. Cellular trafficking determines the exon skipping activity of Pip6α-PMO in mdx skeletal and cardiac muscle cells. Nucleic Acids Res 42, 3207-3217, doi:10.1093/nar/gkt1220 (2014).
27 Nakase, I. et al. Interaction of arginine-rich peptides with membrane-associated proteoglycans is crucial for induction of actin organization and macropinocytosis. Biochemistry 46, 492-501, doi:10.1021/bi0612824 (2007).
28 Chang, Y. S. et al. Stapled alpha-helical peptide drug development: a potent dual inhibitor of MDM2 and MDMX for p53-dependent cancer therapy. Proc Natl Acad Sci USA 110, E3445-3454, doi:10.1073/pnas.1303002110 (2013).
29 Vitiello, L. et al. In vivo delivery of naked antisense oligos in aged mdx mice: analysis of dystrophin restoration in skeletal and cardiac muscle. Neuromuscul Disord 18, 597-605, doi:S0960-8966(08)00141-7 [pii]10.1016/j.nmd.2008.05.011 [doi] (2008).
30 Jearawiriyapaisarn, N., Moulton, H. M., Sazani, P., Kole, R. & Willis, M. S. Long-term improvement in mdx cardiomyopathy after therapy with peptide-conjugated morpholino oligomers. Cardiovasc Res 85, 444-453, doi:cvp335 [pii]10.1093/cvr/cvp335 [doi] (2010).
31 Wu, B. et al. One-year treatment of morpholino antisense oligomer improves skeletal and cardiac muscle functions in dystrophic mdx mice. Mol Ther 19, 576-583, doi:10.1038/mt.2010.288 (2011).
32 Wu, B. et al. Effective rescue of dystrophin improves cardiac function in dystrophin-deficient mice by a modified morpholino oligomer. Proc Natl Acad Sci USA 105, 14814-14819, doi:0805676105 [pii]10.1073/pnas.0805676105 [doi] (2008).
33 Jearawiriyapaisarn, N. et al. Sustained dystrophin expression induced by peptide-conjugated morpholino oligomers in the muscles of mdx mice. Mol Ther 16, 1624-1629, doi:mt2008120 [pii]10.1038/mt.2008.120 [doi] (2008).
34 Betts, C. et al. Pip6-PMO, A New Generation of Peptide-oligonucleotide Conjugates With Improved Cardiac Exon Skipping Activity for DMD Treatment. Molecular therapy. Nucleic acids 1, e38, doi:10.1038/mtna.2012.30 (2012).
35 Ivanova, G. D. et al. Improved cell-penetrating peptide-PNA conjugates for splicing redirection in HeLa cells and exon skipping in mdx mouse muscle. Nucleic Acids Res 36, 6418-6428 (2008).
36 Lautrette, G., Touti, F., Lee, H. G., Dai, P. & Pentelute, B. L. Nitrogen Arylation for Macrocyclization of Unprotected Peptides. J Am Chem Soc 138, 8340-8343, doi:10.1021/jacs.6b03757 (2016).
37 Mendell, J. R. et al. Evidence-based path to newborn screening for Duchenne muscular dystrophy. Ann Neurol 71, 304-313, doi:10.1002/ana.23528 (2012).
38 Hoffman, E. P., Brown, R. H., Jr. & Kunkel, L. M. Dystrophin: the protein product of the Duchenne muscular dystrophy locus. Cell 51, 919-928. (1987).
39 Monaco, A. P., Bertelson, C. J., Liechti-Gallati, S., Moser, H. & Kunkel, L. M. An explanation for the phenotypic differences between patients bearing partial deletions of the DMD locus. Genomics 2, 90-95 (1988).
40 Aartsma-Rus, A. et al. Therapeutic antisense-induced exon skipping in cultured muscle cells from six different DMD patients. Hum Mol Genet 12, 907-914 (2003).
41 Alter, J. et al. Systemic delivery of morpholino oligonucleotide restores dystrophin expression bodywide and improves dystrophic pathology. Nat Med 12, 175-177 (2006).
42 Kinali, M. et al. Local restoration of dystrophin expression with the morpholino oligomer AVI-4658 in Duchenne muscular dystrophy: a single-blind, placebo-controlled, dose-escalation, proof-of-concept study. Lancet Neurol 8, 918-928, doi:S1474-4422(09)70211-X [pii]10.1016/S1474-4422(09)70211-X [doi] (2009).
43 Lu, Q. L. et al. Systemic delivery of antisense oligoribonucleotide restores dystrophin expression in body-wide skeletal muscles. Proc Natl Acad Sci USA 102, 198-203 (2005).
44 van Deutekom, J. C. et al. Local dystrophin restoration with antisense oligonucleotide PRO051. N Engl J Med 357, 2677-2686, doi:357/26/2677 [pii]10.1056/NEJMoa073108 [doi] (2007).
45 Wu, B. et al. Dose-dependent restoration of dystrophin expression in cardiac muscle of dystrophic mice by systemically delivered morpholino. Gene Ther 17, 132-140, doi:gt2009120 [pii]10.1038/gt.2009.120 [doi] (2010).
46 Cirak, S. et al. Exon skipping and dystrophin restoration in patients with Duchenne muscular dystrophy after systemic phosphorodiamidate morpholino oligomer treatment: an open-label, phase 2, dose-escalation study. Lancet 378, 595-605, doi:10.1016/s0140-6736(11)60756-3 (2011).

1. A drug carrying cell penetrating molecule (DCCPM) comprising:
i. a biologically active compound (BAC), and
ii. a cell penetrating agent (CPA),
wherein the BAC and CPA are linked directly or via a bi-functional linker (BFL), the CPA is a stabilized peptide (CPP) which has a conformation imposed upon it by stapling to form a stapled peptide (StaP) or stitching to form a stitched peptide (StiP), the StiP or StaP comprises a cross link or bridge between at least two amino acids of the peptide and the cross link or bridge provides a cyclisation between the at least two amino acids which are not formed by an olefin metathesis.

2. The DCCPM as claimed in claim 1, wherein the cyclisation is achieved by one or more of:
i. condensation of an aldehyde or ketone with a hydrazine or protected hydrazine;
ii. a thiol-ene Michael addition;
iii. a di-sulfide formation;
iv. a Huisgen 1,3 di-polar cycloaddition;
v. a reaction between an amine and carboxylic acid;
vi. a singlet or triplet based carbine reaction; or
vii. a Suzuki or Sonogashira coupling.

3. The DCCPM as claimed in claim 2, wherein in iv) a triazole is formed between an azide or electron deficient nitrile containing amino acid and a propygyl containing amino acid.

4. The DCCPM as claimed in claim 3, wherein the azide is azidolysine.

5. The DCCPM as claimed in claim 3, wherein the propygyl containing amino acid is lysine, glutamic acid or aspartic acid.

6. The DCCPM as claimed in claim 2, wherein in v) a lactam is formed between a free amine containing amino acid and a carboxylic acid containing amino acid, optionally wherein the lactam is formed by cross linking a lysine and glutamic or aspartic acid.

7. (canceled)

8. The DCCPM as claimed in claim 1, wherein each crosslink has a nominal sequential length of from 2-20 atoms.

9. The DCCPM as claimed in claim 1, wherein the stabilized peptide comprises at least one alpha helix, extended 3₁₀-helix or poly (Pro) II helix, or at least one beta sheet or hairpin or turn.

10. (canceled)

11. The DCCPM as claimed in claim 9, wherein the stabilized peptide comprises at least one alpha helix, extended 3₁₀-helix or poly (Pro) II helix and one beta sheet, turn or hairpin.

12. The DCCPM as claimed in claim 1, wherein the BAC is an oligonucleotide (ON).

13.-18. (canceled)

19. The DCCPM as claimed in claim 1, wherein the BAC alters the expression of an endogenous or exogenous gene.

20. (canceled)

21. The DCCPM as claimed in claim 1, wherein the BFL comprises a chemistry selected from the chemistries of Table 6.

22.-29. (canceled)

30. The DCCPM as claimed in claim 1 which is of a size greater than 1.5 KDa.

31. A method for facilitating the uptake of a biologically active compound (BAC) into a cell comprising
conjugating the biologically active compound to a cell penetrating agent (CPA) which is a stabilized peptide having a conformation imposed upon it by stapling to form a stapled peptide (StaP) or stitching to form a stitched peptide (StiP), wherein the StiP or StaP comprises a cross link or bridge between at least two amino acids of the peptide and the cross link or bridge provides a cyclisation between the at least two amino acids which are not formed by an olefin metathesis, directly or via a bi-functional linker (BFL) to form a drug carrying cell penetrating molecule (DCCPM); and
presenting said DCCPM to said cell in a suitable vehicle.

32. A method of treating a disease in a subject comprising administering to the subject a DCCPM as claimed in claim 1, wherein the disease requires alteration of the expression of an endogenous or exogenous gene.

33. The method of claim 32, wherein the disease is neuromuscular disease, a metabolic disease, cancer, an age-related degenerative disease or an acquired viral infection.

34. The method of claim 32, wherein the disease is Duchenne's muscular dystrophy.

35. The method of claim 34, wherein the DCCPM comprises an AO targeting the dystrophin gene.

36. A method comprising:
linking a drug or BAC to a CPP which is a stabilized peptide which has a conformation imposed upon it by stapling to form a stapled peptide (StaP) or stitching to form a stitched peptide (StiP), wherein the StiP or StaP comprises a cross link or bridge between at least two amino acids of the peptide and the cross link or bridge provides a cyclisation between the at least two amino acids which are not formed by an olefin metathesis, and
administering the linked drug or BAC to a subject,
wherein the method improves the bioavailability of the drug or BAC, or introduces the drug or BAC to a site which is refractory to the drug or BAC in its native state.

37. (canceled)

38. The method as claimed in claim 36 wherein the tissue is one of heart, brain, muscle or liver.

39. (canceled)

40. A composition comprising the DCCPM as claimed in claim 1, wherein the composition comprises one or more pharmaceutically acceptable excipients.

41. (canceled)


SEQUENCE LISTING (13 sequences)

SEQ ID NO: 1 - RNA, 30 nt, Artificial Sequence
  Synthetic Polynucleotide
  MISC_FEATURE: Exemplary RNA targeting exon 51 of the dytrophin gene
cuccaacaucaaggaagauggcauuucuag

SEQ ID NO: 2 - protein, 8 aa, Artificial Sequence
  Synthetic Polypeptide
  MISC_FEATURE: Exemplary stabilising peptide
  MOD_RES (4)..(4): N6-diazolysine
  MISC_FEATURE (4)..(8): Crosslink
  MOD_RES (8)..(8): X is artificial amino acid (S)-2-Amino-2- methyl-4-pentynoic acid
RKFKRLFX

SEQ ID NO: 3 - protein, 8 aa, Artificial Sequence
  Synthetic Polypeptide
  MISC_FEATURE: Exemplary stabilising peptide
  MISC_FEATURE (4)..(4): X is an amino acid that is able to be cross linked
  MISC_FEATURE (4)..(8): Cross linked
  MISC_FEATURE (8)..(8): X is an amino acid that is able to be cross linked
RFKXRLFX

SEQ ID NO: 4 - protein, 8 aa, Artificial Sequence
  Synthetic Polypeptide
  MISC_FEATURE: Exemplary stabilising peptide
  MISC_FEATURE (4)..(8): crosslink
RKFERLFK

SEQ ID NO: 5 - protein, 8 aa, Artificial Sequence
  Synthetic Polypeptide
  MISC_FEATURE: Exemplary stabilising peptide
  MISC_FEATURE (3)..(3): X is an amino acid that is able to be cross linked.
  MISC_FEATURE (3)..(8): Cross linked
  MISC_FEATURE (8)..(8): X is an amino acid that is able to be cross linked
RFXKRLFX

SEQ ID NO: 6 - protein, 8 aa, Artificial Sequence
  Synthetic Polypeptide
  MISC_FEATURE: Exemplary stabilising peptide
  MISC_FEATURE (5)..(5): X is an amino acid that is able to be cross linked
  MISC_FEATURE (5)..(8): Cross linked
  MISC_FEATURE (8)..(8): X is an amino acid that is able to be cross linked
RFKRXLFX

SEQ ID NO: 7 - protein, 8 aa, Artificial Sequence
  Synthetic Polypeptide
  MISC_FEATURE: Exemplary stabilising peptide
  MISC_FEATURE (4)..(8): Cross link
RKFERLFK

SEQ ID NO: 8 - protein, 8 aa, Artificial Sequence
  Synthetic Polypeptide
  MISC_FEATURE: Exemplary stabilising peptide
  MISC_FEATURE (4)..(4): X is un-natural amino acid, S-pentenylalanine
  MISC_FEATURE (4)..(8): Cross linked
  MISC_FEATURE (8)..(8): X is un-natural amino acid, S-pentenylalanine
RKFXRLFX

SEQ ID NO: 9 - protein, 8 aa, Artificial Sequence
  Exemplary stabilising sequence
  MISC_FEATURE: Exemplary stabilising sequence
RKFERLFK

SEQ ID NO: 10 - protein, 8 aa, Artificial Sequence
  Synthetic Polypeptide
  MISC_FEATURE: Exemplary stabilising sequence
  MISC_FEATURE (5)..(8): Cross link
RKFRELFK

SEQ ID NO: 11 - protein, 8 aa, Artificial Sequence
  Synthetic Polypeptide
  MISC_FEATURE: Exemplary stabilising sequence
  MISC_FEATURE (3)..(8): Cross link
RKEFRLFK

SEQ ID NO: 12 - protein, 8 aa, Artificial Sequence
  Synthetic Polypeptide
  MISC_FEATURE: Exemplary stabilising peptide
  MOD_RES (5)..(5): (2S)-N-Fmoc-6-azido-hexanoic acid
  MISC_FEATURE (5)..(8): Cross linked
  MOD_RES (8)..(8): X is (S)-N-Fmoc-2-(2′-propynyl)alanine
RKFRKLFX

SEQ ID NO: 13 - protein, 8 aa, Artificial Sequence
  Synthetic Polypeptide
  MISC_FEATURE: Exemplary stabilising peptide
  MOD_RES (3)..(3): (2S)-N-Fmoc-6-azido-hexanoic acid
  MISC_FEATURE (3)..(8): Cross linked
  MISC_FEATURE (8)..(8): X is (S)-N-Fmoc-2-(2′-propynyl)alanine
RKKFRLFX